SEQUENCE LISTING (31 sequences)

SEQ ID NO: 1 - DNA, 1008 bp, Homo sapiens
cacgcacttcacctgggtcgggattctcaggtcatgaacggtcccagccacctccgggcagggcgggtgaggacggggacggggcgtgtccaactggctgtgggctcttgaaacccgagcatggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctgggaacaagacccacaacgctgtgtgcgtcccagggtccccgccggcagagccgcttgggtggctgaccgtcgtcctcctggccgtggccgcctgcgtcctcctcctgacctcggcccagcttggactgcacatctggcagctgaggagtcagtgcatgtggccccgagagacccagctgctgctggaggtgccgccgtcgaccgaagacgccagaagctgccagttccccgaggaagagcggggcgagcgatcggcagaggagaaggggcggctgggagacctgtgggtgtgagcctggccgtcctccggggccaccgaccgcagccagcccctccccaggagctccccaggccgcaggggctctgcgttctgctctgggccgggccctgctcccctggcagcagaagtgggtgcaggaaggtggcagtgaccagcgccctggaccatgcagttc

SEQ ID NO: 2 - DNA, 726 bp, Homo sapiens
atggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctgggaacaagacccacaacgctgtgtgcgtcccagggtccccgccggcagagccgcttgggtggctgaccgtcgtcctcctggccgtggccgcctgcgtcctcctcctgacctcggcccagcttggactgcacatctggcagctgaggagtcagtgcatgtggccccgagagacccagctgctgctggaggtgccgccgtcgaccgaagacgccagaagctgccagttccccgaggaagagcggggcgagcgatcggcagaggagaaggggcggctgggagacctgtgggtgtga

SEQ ID NO: 3 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 4 - DNA, 969 bp, Homo sapiens
  Unsure 954, 963: n may be any nucleotide
ggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctggggaacaagacccacaacgctgtgtgcgtcccagggtccccgccggcagagccgcttgggtggctgaccgtcgtcctcctggccgtggccgcctgcgtctcctcctgacctcggcccagcttggactgcacatctggcagctgaggagtcagtgcatgtggccccgaggtctgtcacagcctggtgcggggaggtgggagcatggctgcctgctgaccgtggcccccctgcatagacccagctgctgctggaggtgccgccgtcgaccgaagacgccagaagctgccagttccccgaggaagagcggggcgagcgatcggcagaggagaaggggcggctgggagacctgtgggtgtgagcctggctgtcctccggggccaccgaccgcagccagcccctccccaggagctccccaggccgcaggggctctgcgttctgctctgggccgggccctgctcccctggcagcagaagtgggtgcaggaaggtggcagtgaccagcgccctggaccatgcagttcggnggccgggtnggccct

SEQ ID NO: 5 - DNA, 19 bp, Artificial sequence
  Misc_feature 1-19: Sequence is synthesized
cacagcacggggcgatggg

SEQ ID NO: 6 - DNA, 18 bp, Artificial Sequence
  Misc_feature 1-18: Sequence is synthesized
gctctgcgttctgctctg

SEQ ID NO: 7 - DNA, 28 bp, Artificial Sequence
  Misc_feature 1-28: Sequence is synthesized
ggcacagcacggggcgatgggcgcgttt

SEQ ID NO: 8 - DNA, 24 bp, Artificial Sequence
  Misc_feature 1-24: Sequence is synthesized
ctggtcactgccaccttcctgcac

SEQ ID NO: 9 - DNA, 17 bp, Artificial Sequence
  Misc_feature 1-17: Sequence is synthesized
cgctgacccaggctgag

SEQ ID NO: 10 - DNA, 27 bp, Artificial sequence
  Misc_feature 1-27: Sequence is synthesized
gaaggtccccgaggcacagtcgataca

SEQ ID NO: 11 - DNA, 40 bp, Artificial sequence
  Misc_feature 1-40: Sequence is synthesized
gaggagtgctgttccgagtgggactgcatgtgtgtccagc

SEQ ID NO: 12 - DNA, 40 bp, Artificial sequence
  Misc_feature 1-40: Sequence is synthesized
agcctgggtcagcgccccaccgggggtcccgggtgcggcc

SEQ ID NO: 13 - DNA, 42 bp, Artificial sequence
  Misc_feature 1-42: Sequence is synthesized
tgtaaaacgacggccagtttctctcagagaaacaagcaaaac

SEQ ID NO: 14 - DNA, 43 bp, Artificial sequence
  Misc_feature 1-43: Sequence is synthesized
caggaaacagctatgaccgaagtggaccaaaggtctatcgcta

SEQ ID NO: 15 - DNA, 1964 bp, Homo sapiens
  Unsure 1857, 1875: n may be any nucleotide
cagctctcatttctccaaaaatgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctagagacttgatttgatctcctcattcccttcagcacatgtagaggtgccagtgggtggattggagggagaagatattcaatttctagagtttgtctgtctacaaaaatcaacacaaacagaactcctctgcacgtgaattttcatctatcatgcctatctgaaagagactcaggggaagagccaaagacttttggttggatctgcagaaatacttcattaatccatgataaaacaaatatggatgacagaggacatgtgcttttcaaagaatctttatctaattcttgaattcatgagtggaaaaatggagttctattcccatggaagatttacctggtatgcaaaaaggatctggggcagtagcctggctttgttctcatattcttgggctgctgtaattcattcttctcatactcccatcttctgagaccctcccaataaaaagtagactgataggatggccacagatatgcctaccataccctactttagatatggtggtgttagaagataaagaacaatctgagaactattggaatagaggtacaagtggcataaaatggaatgtacgctatctggaaatttctcttggttttatcttcctcaggatgcagggtgctttaaaaagccttatcaaaggagtcattccgaaccctcacgtagagctttgtgagaccttactgttggtgtgtgtgtctaaacattgctaattgtaaagaaagagtaaccattagtaatcattaggtttaaccccagaatggtattatcattactggattatgtcatgtaatgatttagtatttttagctagctttccacagtttgcaaagtgctttcgtaaaacagttagcaattctatgaagttaattgggcaggcatttgggggaaaattttagtgatgagaatgtgatagcatagcatagccaactttcctcaactcataggacaagtgactacaagaggcaatgggtagtcccctgcattgcactgtctcagctttagaattgttatttctgctatcgtgttataagactctaaaacttagcgaattcacttttcaggaagcatattcccctttagcccaaggtgagcagagtgaagctacaacagatctttcctttaccagcacacttttttttttttttcctgcctgaatcagggagatccaggatgctgttcaggccaaatcccaaccaaattccccttttcactttgcagggcccatcttagtcaaatgtgctaacttctaaaataataaatagcactaattcaaaatttttggaatcttaaattagctacttgcnggttgcttgttgaaaggnatataatgattacattgtaaacaaatttaaaatatttatggatatttgtgaaaagctgcattatgttaaataatattacatgtaaagct

SEQ ID NO: 16 - protein, 177 aa, Homo sapiens
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 17 - DNA, 38 bp, Artificial sequence
  Misc_feature 1-38: Sequence is synthesized
gaggacaagcatatgttagagactgctaaggagccctg

SEQ ID NO: 18 - DNA, 34 bp, Artificial sequence
  Misc_feature 1-34: Sequence is synthesized
tagcagccggatcctaggagatgaattggggatt

SEQ ID NO: 19 - protein, 72 aa, Artificial sequence
  Misc_feature 1-72: Sequence is synthesized
METGLYHISHISHISHISHISHISHISHISHISHISSERSERGLYHISILEASPASPASPASPLYSHISMET

SEQ ID NO: 20 - DNA, 20 bp, Homo sapiens
ccactgaaaccttggacaga

SEQ ID NO: 21 - DNA, 27 bp, Homo sapiens
cccagttcgggtttctcactgtgttcc

SEQ ID NO: 22 - DNA, 21 bp, Homo sapiens
acagcgttgtgggtcttgttc

SEQ ID NO: 23 - DNA, 1008 bp, Homo sapiens
gaactgcatggtccagggcgctggtcactgccaccttcctgcacccacttctgctgccaggggagcagggcccggcccagagcagaacgcagagcccctgcggcctggggagctcctggggaggggctggctgcggtcggtggccccggaggacggccaggctcacacccacaggtctcccagccgccccttctcctctgccgatcgctcgccccgctcttcctcggggaactggcagcttctggcgtcttcggtcgacggcggcacctccagcagcagctgggtctctcggggccacatgcactgactcctcagctgccagatgtgcagtccaagctgggccgaggtcaggaggaggacgcaggcggccacggccaggaggacgacggtcagccacccaagcggctctgccggcggggaccctgggacgcacacagcgttgtgggtcttgttcccagggaacacagtgagaaacccgaactgggtgcagtctgtccaaggtttgcagtggccttcgtggcccccggagaaggtccccgaggcacagtcgatacactggaagccaaaactgaatttcccctgggactgtaccccctggcctgggggacaagggtggtgccggcaggtcgtgcagcaagggtctccgcagtggaattcaggctggacacacatgcagtcccactcggaacagcactcctcgcccgggtaatcgcggcagcagcgcgtcgtgtgaacccggcagcagcgcgcgtccgttcccgtcccaagcaggaggcgcccagggccgcacccgggacccccggtggggcgctgacccaggctgagcgcgcacagcagcgccaggccgcacagggcccgaaacgcgcccatcgccccgtgctgtgccatgctcgggtttcaagagcccacagccagttggacacgccccgtccccgtcctcacccgccctgcccggaggtggctgggaccgttcatgacctgagaatcccgacccaggtgaagtgcgtg

SEQ ID NO: 24 - protein, 228 aa, Mus musculus
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHLTVIFLVMAACIFFLTTVQLGLHIWQLRRQHMCPRETQPFAEVQLSAEDACSFQFPEEERGEQTEEKCHLGGRWP

SEQ ID NO: 25 - DNA, 969 bp, Homo sapiens
  Unsure 7,16: n may be any nucleotide
agggccnacccggccnccgaactgcatggtccagggcgctggtcactgccaccttcctgcacccacttctgctgccaggggagcagggcccggcccagagcagaacgcagagcccctgcggcctggggagctcctggggaggggctggctgcggtcggtggccccggaggacagccaggctcacacccacaggtctcccagccgccccttctcctctgccgatcgctcgccccgctcttcctcggggaactggcagcttctggcgtcttcggtcgacggcggcacctccagcagcagctgggtctatgcaggggggccacggtcagcaggcagccatgctcccacctccccgcaccaggctgtgacagacctcggggccacatgcactgactcctcagctgccagatgtgcagtccaagctgggccgaggtcaggaggagacgcaggcggccacggccaggaggacgacggtcagccacccaagcggctctgccggcggggaccctgggacgcacacagcgttgtgggtcttgttccccagggaacacagtgagaaacccgaactgggtgcagtctgtccaaggtttgcagtggccttcgtggcccccggagaaggtccccgaggcacagtcgatacactggaagccaaaactgaatttcccctgggactgtaccccctggcctgggggacaagggtggtgccggcaggtcgtgcagcaagggtctccgcagtggaattcaggctggacacacatgcagtcccactcggaacagcactcctcgcccgggtaatcgcggcagcagcgcgtcgtgtgaacccggcagcagcgcgcgtccgttcccgtcccaagcaggaggcgcccagggccgcacccgggacccccggtggggcgctgacccaggctgagcgcgcacagcagcgccaggccgcacagggcccgaaacgcgcccatcgccccgtgctgtgcc

SEQ ID NO: 26 - protein, 317 aa, Homo sapiens
  Unsure 313, 316: Xaa may be any amino acid
MGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGEQDPQRCVRPRVPAGRAAWVADRRPPGRGRLRLLLTSAQLGLHIWQLRSQCMWPRGLSQPGAGRWEHGCLLTVAPLHRPSCCWRCRRRPKTPEAASSPRKSGASDRQRRRGGWETCGCEPGCPPGPPTAASPSPGAPQAAGALRSALGRALLPWQQKWVQEGGSDQRPGPCSSXAGXA

SEQ ID NO: 27 - DNA, 1964 bp, Homo sapiens
  Unsure 90, 108: n may be any nucleotide
agctttacatgtaatattatttaacataatgcagcttttcacaaatatccataaatattttaaatttgtttacaatgtaatcattatatncctttcaacaagcaaccngcaagtagctaatttaagattccaaaaattttgaattagtgctatttattattttagaagttagcacatttgactaagatgggccctgcaaagtgaaaaggggaatttggttgggatttggcctgaacagcatcctggatctccctgattcaggcaggaaaaaaaaaaaaaagtgtgctggtaaaggaaagatctgttgtagcttcactctgctcaccttgggctaaaggggaatatgcttcctgaaaagtgaattcgctaagttttagagtcttataacacgatagcagaaataacaattctaaagctgagacagtgcaatgcaggggactacccattgcctcttgtagtcacttgtcctatgagttgaggaaagttggctatgctatgctatcacattctcatcactaaaattttcccccaaatgcctgcccaattaacttcatagaattgctaactgttttacgaaagcactttgcaaactgtggaaagctagctaaaaatactaaatcattacatgacataatccagtaatgataataccattctggggttaaacctaatgattactaatggttactctttctttacaattagcaatgtttagacacacacaccaacagtaaggtctcacaaagctctacgtgagggttcggaatgactcctttgataaggctttttaaagcaccctgcatcctgaggaagataaaaccaagagaaatttccagatagcgtacattccattttatgccacttgtacctctattccaatagttctcagattgttctttatcttctaacaccaccatatctaaagtagggtatggtaggcatatctgtggccatcctatcagtctactttttattgggagggtctcagaagatgggagtatgagaagaatgaattacagcagcccaagaatatgagaacaaagccaggctactgccccagatcctttttgcataccaggtaaatcttccatgggaatagaactccatttttccactcatgaattcaagaattagataaagattctttgaaaagcacatgtcctctgtcatccatatttgttttatcatggattaatgaagtatttctgcagatccaaccaaaagtctttggctcttcccctgagtctctttcagataggcatgatagatgaaaattcacgtgcagaggagttctgtttgtgttgatttttgtagacagacaaactctagaaattgaatatcttctccctccaatccacccactggcacctctacatgtgctgaagggaatgaggagatcaaatcaagtctctaggagatgaattggggatttgctagtaaaatgataccccagtatgtattattttttagaacctgatgctcagagttgaatatcaagtctatggtgtccccaacatgcaattcataagtccctcctacattttggattttagatttgtttgttagagtttgtatcatgtctttgtttttatacagccgcacctcaaaaggagctacatcattgtagtttgcattgggagccacttggccataaattaaatataagccattctgaagtatctccagcttccagtcagacaccttattcacgcaaggaggttcagaagatgccatttgccattttgagggtaatggtccaaacttagccatacagggctccttagcagtctctaattggagaaaaataaagattagccaactgaaggagcaaagaaatagcaacataactattgagcaaaagagccacagcttccaggatgatctctgagctccttgagttcttgaatggcttaaaggcatattttccaagtggctcaaacacatttttggagaaatgagagctg

SEQ ID NO: 28 - protein, 150 aa, Homo sapiens
PSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 29 - protein, 164 aa, Homo sapiens
GPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQCGIFELKENDRIFVSVTNEHLIDMDHEASFFGAFLVG

SEQ ID NO: 30 - protein, 140 aa, Homo sapiens
ELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 31 - protein, 147 aa, Homo sapiens
STLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

(19) United States
(12) Patent Application Publication
(10) Pub. No.: US 2005/0069983 A1
(43) Pub. Date: Mar. 31, 2005

(54) Novel tumor necrosis factor receptor homolog and nucleic acids encoding the same

(75) Inventors: Avi J. Ashkenazi, San Mateo, CA (US); Audrey Goddard, San Francisco, CA (US); Austin Gurney, Belmont, CA (US); Scot A. Marsters, San Carlos, CA (US); Robert M. Pitti, El Cerrito, CA (US); William I. Wood, Hillsborough, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/959,537

(22) Filed: Oct. 6, 2004

Related U.S. Application Data
(63) Continuation of application No. 10/116,378, filed on Apr. 4, 2002.
Application No. 10/116,378 is a continuation of application No. 09/247,225, filed on Feb. 9, 1999, now abandoned.
(60) Provisional application No. 60/074,087, filed on Feb. 9, 1998.

(51) Int. Cl. C07K 14/715; C12P 21/04

(52) U.S. Cl. 435/69.1; 435/320.1; 435/325; 530/350; 536/23.5

(57) ABSTRACT

The present invention is directed to novel polypeptides having homology to members of the tumor necrosis factor receptor family and to nucleic acid molecules encoding those polypeptides. Also provided herein are vectors and host cells comprising those nucleic acid sequences, chimeric polypeptide molecules comprising the polypeptides of the present invention fused to heterologous polypeptide sequences, antibodies which bind to the polypeptides of the present invention and to methods for producing the polypeptides of the present invention.

### RELATED APPLICATIONS

This is a non-provisional application filed under 37 CFR 1.53(b) claiming priority under Section 119(e) to provisional application No. 60/074,087 filed Feb. 9, 1998, the contents of which are incorporated herein by reference.

### FIELD OF THE INVENTION

The present invention relates generally to the identification and isolation of novel DNA and to the recombinant production of novel polypeptides having homology to tumor necrosis factor receptor, designated herein as “PRO364” polypeptides.

### BACKGROUND OF THE INVENTION

Control of cell numbers in mammals is believed to be determined, in part, by a balance between cell proliferation and cell death. One form of cell death, sometimes referred to as necrotic cell death, is typically characterized as a pathologic form of cell death resulting from some trauma or cellular injury. In contrast, there is another, “physiologic” form of cell death which usually proceeds in an orderly or controlled manner. This orderly or controlled form of cell death is often referred to as “apoptosis” [see, e.g., Barr et al., Bio/Technology, 12:487-493 (1994); Steller et al., Science, 267:1445-1449 (1995)]. Apoptotic cell death naturally occurs in many physiological processes, including embryonic development and clonal selection in the immune system [Itoh et al., Cell, 66:233-243 (1991)]. Decreased levels of apoptotic cell death have been associated with a variety of pathological conditions, including cancer, lupus, and herpes virus infection [Thompson, Science, 267:1456-1462 (1995)]. Increased levels of apoptotic cell death may be associated with a variety of other pathological conditions, including AIDS, Alzheimer's disease, Parkinson's disease, amyotrophic lateral sclerosis, multiple sclerosis, retinitis pigmentosa, cerebellar degeneration, aplastic anemia, myocardial infarction, stroke, reperfusion injury, and toxin-induced liver disease [see, Thompson, supra].

Apoptotic cell death is typically accompanied by one or more characteristic morphological and biochemical changes in cells, such as condensation of cytoplasm, loss of plasma membrane microvilli, segmentation of the nucleus, degradation of chromosomal DNA or loss of mitochondrial function. A variety of extrinsic and intrinsic signals are believed to trigger or induce such morphological and biochemical cellular changes [Raff, Nature, 356:397-400 (1992); Steller, supra; Sachs et al., Blood, 82:15 (1993)]. For instance, they can be triggered by hormonal stimuli, such as glucocorticoid hormones for immature thymocytes, as well as withdrawal of certain growth factors [Watanabe-Fukunaga et al., Nature, 356:314-317 (1992)]. Also, some identified oncogenes such as myc, rel, and E1A, and tumor suppressors, like p53, have been reported to have a role in inducing apoptosis. Certain chemotherapy drugs and some forms of radiation have likewise been observed to have apoptosis-inducing activity [Thompson, supra].

Various molecules, such as tumor necrosis factor-α (“TNF-α”), tumor necrosis factor-β (“TNF-β” or “lymphotoxin-α”), lymphotoxin-β (“LT-β”), CD30 ligand, CD27 ligand, CD40 ligand, OX-40 ligand, 4-1BB ligand, Apo-1 ligand (also referred to as Fas ligand or CD95 ligand), and Apo-2 ligand (also referred to as TRAIL) have been identified as members of the tumor necrosis factor (“TNF”) family of cytokines [See, e.g., Gruss and Dower, Blood, 85:3378-3404 (1995); Pitti et al., J. Biol. Chem., 271:12687-12690 (1996); Wiley et al., Immunity, 1:673-682 (1995); Browning et al., Cell, 32:847-856 (1993); Armitage et al. Nature, 357:80-82 (1992), WO 97/01633 published Jan. 16, 1997; WO 97/25428 published Jul. 17, 1997]. Among these molecules, TNF-α, TNF-β, CD30 ligand, 4-1BB ligand, Apo-1 ligand, and Apo-2 ligand (TRAIL) have been reported to be involved in apoptotic cell death. Both TNF-α and TNF-β have been reported to induce apoptotic death in susceptible tumor cells [Schmid et al., Proc Natl Acad Sci., 83:1881 (1986); Dealtry et al., Eur. J. Immunol., 17:689 (1987)]. Zheng et al. have reported that TNF-α is involved in post-stimulation apoptosis of CD8-positive T cells [Zheng et al., Nature, 377:348-351 (1995)]. Other investigators have reported that CD30 ligand may be involved in deletion of self-reactive T cells in the thymus [Amakawa et al., Cold Spring Harbor Laboratory Symposium on Programmed Cell Death, Abstr. No. 10, (1995)].

Mutations in the mouse Fas/Apo-1 receptor or ligand genes (called lpr and gld⁻, respectively) have been associated with some autoimmune disorders, indicating that Apo-1 ligand may play a role in regulating the clonal deletion of self-reactive lymphocytes in the periphery [Krammer et al., Curr. Op. Immunol. 6:279-289 (1994); Nagata et al., Science, 267:1449-1456 (1995)]. Apo-1 ligand is also reported to induce post-stimulation apoptosis in CD4-positive T lymphocytes and in B lymphocytes, and may be involved in the elimination of activated lymphocytes when their function is no longer needed [Krammer et al., supra; Nagata et al., supra]. Agonist mouse monoclonal antibodies specifically binding to the Apo-1 receptor have been reported to exhibit cell killing activity that is comparable to or similar to that of TNF-α [Yonehara et al., J. Exp. Med., 169:1747-1756 (1989)].

Induction of various cellular responses mediated by such TNF family cytokines is believed to be initiated by their binding to specific cell receptors. Two distinct TNF receptors of approximately 55-kDa (TNFR1) and 75-kDa (TNFR2) have been identified [Hohman et al., J. Bio. Chem., 264:14927-14934 (1989); Brockhaus et al., Proc. Natl. Acad. Sci., 87:3127-3131 (1990); EP 417, 563, published Mar. 20, 1991] and human and mouse cDNAs corresponding to both receptor types have been isolated and characterized [Loetscher et al., Cell, 61:351 (1990); Schall et al., Cell, 61:361 (1990); Smith et al., Science, 74:1019-1023 (1990); Lewis et al., Proc. Natl. Acad. Sci., 88:2830-2834 (1991); Goodwin et al., Mol. Cell. Biol., 11:3020-3026 (1991)]. Extensive polymorphisms have been associated with both TNF receptor genes [see, e.g., Takao et al., Immunogenetics., 37:199-203 (1993)]. Both TNFRs share the typical structure of cell surface receptors including extracellular, transmembrane and intracellular regions. The extracellular portions of both receptors are found naturally also as soluble TNF-binding proteins [Nophar, Y. et al., EMBO J., 9:3269 (1990); and Kohno, T. et al., Proc. Natl. Acad. Sci U.S.A., 87:8331 (1990)]. More recently, the cloning of recombinant soluble TNF receptors was reported by Hale et al. [J. Cell. Biochem. Supplement 15F, 1991, p. 113 (P424)].

The extracellular portion of type 1 and type 2 TNFRs (TNFR1 and TNFR2) contains a repetitive amino acid sequence pattern of four cysteine-rich domains (CRDs) designated 1 through 4, starting from the NH₂-terminus. Each CRD is about 40 amino acids long and contains 4 to 6 cysteine residues at positions which are well conserved [Schall et al., supra; Loetscher et al., supra; Smith et al., supra; Nophar et al., supra; Kohno et al., supra]. In TNFR1, the approximate boundaries of the four CRDs are as follows: CRD1-amino acids 14 to about 53; CRD2-amino acids from about 54 to about 97; CRD3-amino acids from about 98 to about 138; CRD4− amino acids from about 139 to about 167. In TNFR2, CRD1 includes amino acids 17 to about 54; CRD2− amino acids from about 55 to about 97; CRD3− amino acids from about 98 to about 140; and CRD4− amino acids from about 141 to about 179 [Banner et al., Cell, 73:431-435 (1993)]. The potential role of the CRDs in ligand binding is also described by Banner et al., supra.

A similar repetitive pattern of CRDs exists in several other cell-surface proteins, including the p75 nerve growth factor receptor (NGFR) [Johnson et al., Cell 47:545 (1986); Radeke et al., Nature, 325:593 (1987)], the B cell antigen CD40 [Stamenkovic et al., EMBO J., 8:1403 (1989)], the T cell antigen OX40 [Mallet et al., EMBO J., 9:1063 (1990)] and the Fas antigen [Yonehara et al., supra and Itoh et al., Cell, 66:233-243 (1991)]. CRDs are also found in the soluble TNFR (sTNFR)-like T2 proteins of the Shope and myxoma poxviruses [Upton et al., Virology, 160:20-29 (1987); Smith et al., Biochem. Biophys. Res. Commun., 176:335 (1991); Upton et al., Virology, 18:370 (1991)].

Optimal alignment of these sequences indicates that the positions of the cysteine residues are well conserved. These receptors are sometimes collectively referred to as members of the TNF/NGF receptor superfamily. Recent studies on p75NGFR showed that the deletion of CRD1 [Welcher, A. A. et al., Proc. Natl. Acad. Sci. USA, 88:159-163 (1991)] or a 5-amino acid insertion in this domain [Yan, H. and Chao, M. V., J. Biol. Chem 266:12099-12104 (1991)] had little or no effect on NGF binding [Yan, H. and Chao, M. V., supra]. p75 NGFR contains a proline-rich stretch of about 60 amino acids, between its CRD4 and transmembrane region, which is not involved in NGF binding [Peetre, C. et al., Eur. J. Hematol., 41:414-419 (1988); Seckinger, P. et al., J. Biol. Chem., 264:11966-11973. (1989); Yan, H. and Chao, M. V., supra]. A similar proline-rich region is found in TNFR2 but not in TNFR1.

The TNF family ligands identified to date, with the exception of lymphotoxin-α, are type II transmembrane proteins, whose C-terminus is extracellular. In contrast, most receptors in the TNF receptor (TNFR) family identified to date are type I transmembrane proteins. In both the TNF ligand and receptor families, however, homology identified between family members has been found mainly in the extracellular domain (“ECD”). Several of the TNF family cytokines, including TNF-α, Apo-1 ligand and CD40 ligand, are cleaved proteolytically at the cell surface; the resulting protein in each case typically forms a homotrimeric molecule that functions as a soluble cytokine. TNF receptor family proteins are also usually cleaved proteolytically to release soluble receptor ECDs that can function as inhibitors of the cognate cytokines.

Recently, other members of the TNFR family have been identified. Such newly identified members of the TNFR family include CAR1, HVEM and osteoprotegerin (OPG) [Brojatsch et al., Cell, 87:845-855 (1996); Montgomery et al., Cell, 87:427-436 (1996); Marsters et al., J. Biol. Chem., 977:14029-14032 (1997); Simonet et al., Cell, 89:309-319 (1997)]. Unlike other known TNFR-like molecules, Simonet et al., supra, report that OPG contains no hydrophobic transmembrane-spanning sequence.

Moreover, a new member of the TNF/NGF receptor family has been identified in mouse, a receptor referred to as “GITR” for “glucocorticoid-induced tumor-necrosis factor receptor family-related gene” [Nocentini et al., Proc Natl. Acad. Sci. USA 94:6216-6221 (1997)]. The mouse GITR receptor is a 228 amino acid type I transmembrane protein that is expressed in normal mouse T lymphocytes from thymus, spleen and lymph nodes. Expression of the mouse GITR receptor was induced in T lymphocytes upon activation with anti-CD3 antibodies, Con A or phorbol 12-myristate 13-acetate. It was speculated by the authors that the mouse GITR receptor was involved in the regulation of T cell receptor-mediated cell death.

In Marsters et al., Curr. Biol., 6:750 (1996), investigators describe a full length native sequence human polypeptide, called Apo-3, which exhibits similarity to the TNFR family in its extracellular cysteine-rich repeats and resembles TNFR1 and CD95 in that it contains a cytoplasmic death domain sequence [see also Marsters et al., Curr. Biol., 6:1669 (1996)]. Apo-3 has also been referred to by other investigators as DR3, wsl-1 and TRAMP [Chinnaiyan et al., Science, 214:990 (1996); Kitson et al., Nature, 384:372 (1996); Bodmer et al., Immunity, 6:79 (1997)].

Pan et al. have disclosed another TNF receptor family member referred to as “DR4” [Pan et al., Science, 227:111-113 (1997)]. The DR4 was reported to contain a cytoplasmic death domain capable of engaging the cell suicide apparatus. Pan et al. disclose that DR4 is believed to be a receptor for the ligand known as Apo-2 ligand or TRAIL.

In Sheridan et al., Science, 277:818-821 (1997) and Pan et al., Science, 277:815-818 (1997), another molecule believed to be a receptor for the Apo-2 ligand (TRAIL) is described. That molecule is referred to as DR5 (it has also been alternatively referred to as Apo-2). Like DR4, DR5 is reported to contain a cytoplasmic death domain and be capable of signaling apoptosis.

In Sheridan et al., supra, a receptor called DcR1 (or alternatively, Apo-2DcR) is disclosed as being a potential decoy receptor for Apo-2 ligand (TRAIL). Sheridan et al. report that DcR1 can inhibit Apo-2 ligand function in vitro. See also, Pan et al. supra, for disclosure on the decoy receptor referred to as TRID.

For a review of the TNF family of cytokines and their receptors, see Gruss and Dower, supra.

As presently understood, the cell death program contains at least three important elements—activators, inhibitors, and effectors; in C. elegans, these elements are encoded respectively by three genes, Ced-4, Ced-9 and Ced-3 [Steller, Science, 267:1445 (1995); Chinnaiyan et al., Science, 275:1122-1126 (1997); Wang et al., Cell, 90:1-20 (1997)]. Two of the TNFR family members, TNFR1 and Fas/Apol (CD95), can activate apoptotic cell death [Chinnaiyan and Dixit, Current Biology, 6:555-562 (1996); Fraser and Evan, Cell; 85:781-784 (1996)]. TNFR1 is also known to mediate activation of the transcription factor, NF-κB [Tartaglia et al., Cell, 74:845-853 (1993); Hsu et al., Cell, 84:299-308 (1996)). In addition to some ECD homology, these two receptors share homology in their intracellular domain (ICD) in an oligomerization interface known as the death domain [Tartaglia et al., supra; Nagata, Cell, 88:355 (1997)]. Death domains are also found in several metazoan proteins that regulate apoptosis, namely, the Drosophila protein, Reaper, and the mammalian proteins referred to as FADD/MORT1, TRADD, and RIP (Cleaveland and Ihle, Cell, 81:479-482 (1995)].

Upon ligand binding and receptor clustering, TNFR1 and CD95 are believed to recruit FADD into a death-inducing signalling complex. CD95 purportedly binds FADD directly, while TNFR1 binds FADD indirectly via TRADD [Chinnaiyan et al., Cell, 81:505-512 (1995); Boldin et al., J. Biol. Chem., 270:387-391 (1995); Hsu et al., supra; Chinnaiyan et al., J. Biol. Chem., 271:4961-4965 (1996)). It has been reported that FADD serves as an adaptor protein which recruits the Ced-3-related protease, MACHα/FLICE (caspase 8), into the death signalling complex [Boldin et al., Cell, 85:803-815 (1996); Muzio et al., Cell, 85:817-827 (1996)]. MACHα/FLICE appears to be the trigger that sets off a cascade of apoptotic proteases, including the interleukin-1β converting enzyme (ICE) and CPP32/Yama, which may execute some critical aspects of the cell death programme [Fraser and Evan, supra].

It was recently disclosed that programmed cell death involves the activity of members of a family of cysteine proteases-related to the C. elegans cell death gene, ced-3, and to the mammalian IL-1-converting enzyme, ICE. The activity of the ICE and CPP32/Yama proteases can be inhibited by the product of the cowpox virus gene, crmA [Ray et al., Cell, 69:597-604 (1992); Tewari et al., Cell, 81:801-809 (1995)]. Recent studies show that CrmA can inhibit TNFR1- and CD95-induced cell death [Enari et al., Nature, 375:78-81 (1995); Tewari et al., J. Biol. Chem., 270:3255-3260 (1995)].

As reviewed recently by Tewari et al., TNFR1, TNFR2 and CD40 modulate the expression of proinflammatory and costimulatory cytokines, cytokine receptors, and cell adhesion molecules through activation of the transcription factor, NF-κB [Tewari et al., Curr. Op. Genet. Develop., 6:39-44 (1996)). NF-κB is the prototype of a family of dimeric transcription factors whose subunits contain conserved Rel regions [Verma et al., Genes Develop., 9:2723-2735 (1996); Baldwin, Ann. Rev. Immunol., 14:649-681 (1996)]. In its latent form, NF-κB is complexed with members of the IκB inhibitor family; upon inactivation of the IκB in response to certain stimuli, released NF-κB translocates to the nucleus where it binds to specific DNA sequences and activates gene transcription.

### SUMMARY OF THE INVENTION

Applicants have-identified a cDNA clone that encodes a novel polypeptide having certain sequence identity to previously-described tumor necrosis factor receptor protein(s), wherein the polypeptide is designated in the present application as “PRO364”.

In one embodiment, the invention provides an isolated nucleic acid molecule comprising DNA encoding a PRO364 polypeptide. In certain aspects, the isolated nucleic acid comprises DNA encoding the PRO364 polypeptide having amino acid residues 1 to 241, 26 to 241, 1-161 or 26-161 of FIG. 2A (SEQ ID NO:3), or is complementary to such encoding nucleic acid sequences, and remains stably bound to it under at least moderate, and optionally, under high stringency conditions. The isolated nucleic acid sequence may comprise the cDNA insert of the vector deposited on Nov. 7, 1997 as ATCC 209436 which includes the nucleotide sequence encoding PRO364.

In another embodiment, the invention provides a vector comprising DNA encoding a PRO364 polypeptide. A host cell comprising such a vector is also provided. By way of example, the host cells may be CHO cells, E. coli, or yeast. A process for producing PRO364 polypeptides is further provided and comprises culturing host cells under conditions suitable for expression of PRO364 and recovering PRO364 from the cell culture.

In another embodiment, the invention provides isolated PRO364 polypeptide. In particular, the invention provides isolated native sequence PRO364 polypeptide, which in one embodiment, includes an amino acid sequence comprising residues 1 to 241 of FIG. 2A (SEQ ID NO:3). Additional embodiments of the present invention are directed to isolated extracellular domain sequences of a PRO364 polypeptide comprising amino acids 1-161, 26-161 or 26-241 of the amino acid sequence shown in FIG. 2A (SEQ ID NO:3), or fragments thereof. Optionally, the PRO364 polypeptide is obtained or is obtainable by expressing the polypeptide encoded by the cDNA insert of the vector deposited on Nov. 7, 1997 as ATCC 209436.

In another embodiment, the invention provides chimeric molecules comprising a PRO364 polypeptide or extracellular domain sequence or other fragment thereof fused to a heterologous polypeptide or amino acid sequence. An example of such a chimeric molecule comprises a PRO364 polypeptide fused to an epitope tag sequence or a Fc region of an immunoglobulin.

In another embodiment, the invention provides an antibody which specifically binds to a PRO364 polypeptide or extracellular domain thereof. Optionally, the antibody is a monoclonal antibody.

In a still further embodiment, the invention provides diagnostic and therapeutic methods using the PRO364 polypeptide or DNA encoding the PRO364 polypeptide.

### BRIEF DESCRIPTION OF THE DRAWINGS

FIG. 1 shows a nucleotide sequence (SEQ ID NO:1) containing the nucleotide sequence (SEQ ID NO:2) of a native sequence PRO364 cDNA (nucleotides 121-843), wherein the nucleotide sequence (SEQ ID NO:1) is a clone designated herein as “UNQ319” and/or “DNA47365-1206”. Also presented is the position of the initiator methionine residue as well as the position of three oligonucleotide primers designated “47365.tm.f”, “47365.tm.p” and “47365.tm.r” as underlined. The putative transmembrane domain of the protein is encoded by nucleotides 604-660 in the figure.

FIG. 2A shows the amino acid sequence (SEQ ID NO:3) derived from nucleotides 121-843 of the nucleotide sequence shown in FIG. 1. A potential transmembrane domain exists between and including amino acids 162 to 180 in the figure. FIG. 2B shows an alignment of the amino acid sequence of PRO364 with murine GITR. The predicted CRDs are indicated, as is the putative transmembrane domain (TM). Identical residues are shaded, and the potential N-linked glycosylation sites are indicated with bullets.

FIGS. 3A-C show a consensus nucleotide sequence designated “<consen01>”.

FIG. 4 shows the “<consen01>” consensus nucleotide sequence shown in FIGS. 3A-C designated in the present application as DNA44825 (SEQ ID NO:4). Also presented is the position of the oligonucleotide primers designated “44825.GITR.f”, “44825.f1”, “44825.GITR.p”, “44825.r2”, “44825.p1”, “44825.GITR.r”, “44825.f2” and “44825.r1” as underlined.

FIGS. 5A-B show the encoding nucleotide sequence (SEQ ID NO:15) and deduced amino acid sequence (SEQ ID NO:16) of a cDNA clone designated herein as DNA19355-1150.

FIG. 6 shows a comparison of amino acid sequences of the polypeptide encoded by DNA19355-1150(DNA19355) with several members of the TNF cytokine family, including human Apo-2L, Fas/Apol-ligand, TNF-alpha and Lymphotoxin-α.

FIG. 7 illustrates the relative mRNA expression of PRO364 in various human cells and tissues, as determined by quantitative reverse-transcriptase PCR.

FIG. 8 illustrates the relative mRNA expression of PRO364 in primary human T cells and monocytes (treated with anti-CD3 antibody, PHA or LPS), as determined by quantitative reverse-transcriptase PCR.

FIG. 9 shows the results of a co-precipitation assay described in Example 10 below. The autoradiograph of the SDS-PAGE gel revealed the PRO364-IgG molecule bound to the radioiodinated DNA19355 polypeptide. Binding was not observed for the other immunoadhesin constructs identified.

FIG. 10A shows the results of FACS analysis of transfected 293 cells assayed for binding to the identified receptors or ligand immunoadhesin constructs.

FIG. 10B shows the results of FACS analysis of HUVEC cells assayed for binding to the identified immunoadhesin constructs.

FIG. 11 shows the results of a luciferase activity assay conducted to demonstrate NF-κB activation by the DNA19355 ligand/PRO364 receptor.

FIG. 12 shows the results of a luciferase activity assay conducted to determine the role of certain intracellular signaling molecules in NF-κB activation by the DNA19355 ligand/PRO364 receptor.

FIG. 13 is a graph showing the effect of a PRO364/DNA19355 ligand on AICD in the human Jurkat T cell line.

### DETAILED DESCRIPTION OF THE PREFERRED EMBODIMENTS

### I. Definitions

The terms “PRO364 polypeptide” and “PRO364” when used herein encompass native sequence PRO364 and PRO364 polypeptide variants (which are further defined herein). The PRO364 polypeptides may be isolated from a variety of sources, such as from human tissue types or from another source, or prepared by recombinant or synthetic methods.

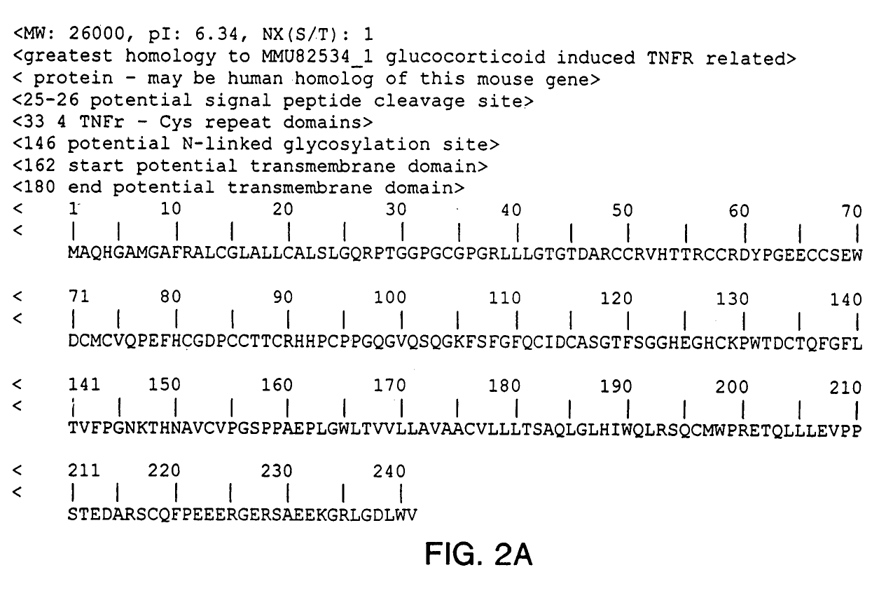

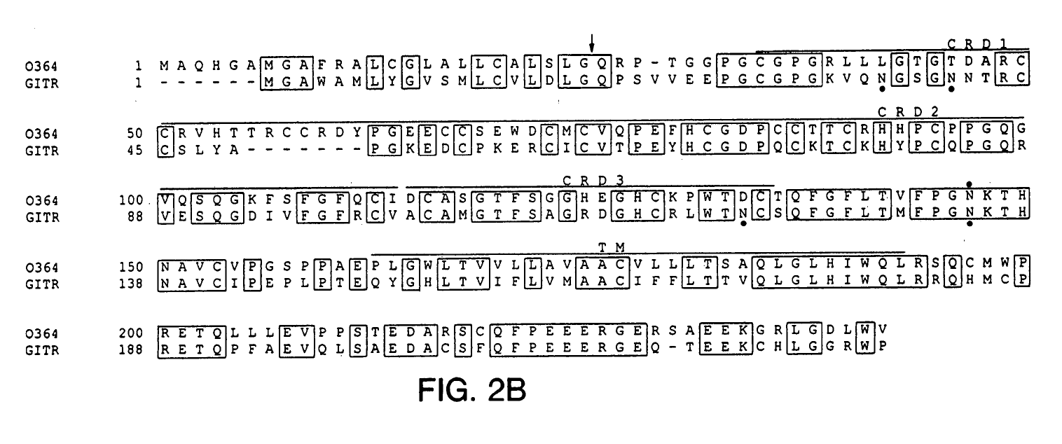

A “native sequence PRO364 polypeptide” comprises a polypeptide having the same amino acid sequence as a PRO364 polypeptide derived from nature. Such native sequence PRO364 polypeptide can be isolated from nature or can be produced by recombinant or synthetic means. The term “native sequence PRO364 polypeptide” specifically encompasses naturally-occurring truncated or secreted forms of a PRO364 polypeptide (e.g., soluble forms containing for instance, an extracellular domain sequence), naturally-occurring variant forms (e.g., alternatively spliced forms) and naturally-occurring allelic variants of a PRO364 polypeptide. In one embodiment of the invention, the native sequence PRO364 polypeptide is a mature or full-length native sequence PRO364 polypeptide comprising amino acids 1 to 241 of FIG. 2A (SEQ ID NO:3). Additional embodiments are directed to PRO364 polypeptide comprising amino acids 26-241 of FIG. 2A (SEQ ID NO:3). In yet another embodiment of the invention, the native sequence PRO364 polypeptide is an extracellular domain sequence of the full-length PRO364 protein, wherein the putative-transmembrane domain of the full-length PRO364 protein includes amino acids 162-180 of the sequence shown in FIG. 2A (SEQ ID NO:3). Thus, additional embodiments of the present invention are directed to polypeptides comprising amino acids 1-161 or 26-161 of the amino acid sequence shown in FIG. 2A (SEQ ID NO:3). Optionally, the PRO364 polypeptide is obtained or obtainable by expressing the polypeptide encoded by the cDNA insert of the vector DNA47365-1206 deposited on Nov. 7, 1997 as ATCC 209436.

The “PRO364 extracellular domain” or “PRO364 ECD” refers to a form of the PRO364 polypeptide which is essentially free of the transmembrane and cytoplasmic domains of the PRO364 polypeptide. Ordinarily, PRO364 ECD will have less than 1% of such transmembrane and/or cytoplasmic domains and preferably, will have less than 0.5% of such domains. Optionally, PRO364 polypeptide ECD will comprise amino acid residues 1-161 of FIG. 2A (SEQ ID NO:3). Included are deletion variants or fragments of the full length or ECD in which one or more amino acids are deleted from the N- or C-terminus. Preferably, such deletion variants or fragments possess a desired activity, such as described herein. It will be understood that any transmembrane domain identified for the PRO364 polypeptide of the present invention is identified pursuant to criteria routinely employed in the art for identifying that type of hydrophobic domain. The exact boundaries of a transmembrane domain may vary but most likely by no more than about 5 amino acids at either end of the domain as initially identified. Accordingly, the PRO364 polypeptide ECD may optionally comprise amino acids Y to X of FIG. 2A (SEQ ID NO:3), wherein Y is any one of amino acid residues 1 to 26 and X is any one of amino acid residues 157 to 167 of FIG. 2A (SEQ ID NO:3).

“PRO364 variant” means a PRO364 polypeptide as defined below having at least about 80% amino acid sequence identity with the PRO364 polypeptide having the deduced amino acid sequence shown in FIG. 2A (SEQ ID NO:3) for a full-length native sequence PRO364 polypeptide or a PRO364 ECD sequence. Such PRO364 polypeptide variants include, for instance, PRO364 polypeptides wherein one or more amino acid residues are added, or deleted, at the N- or C-terminus of the sequence of FIG. 2A (SEQ ID NO:3). Ordinarily, a PRO364 polypeptide variant will have at least about 80% amino acid sequence identity, preferably at least about 85% amino acid sequence identity, more preferably at least about 90% amino acid sequence identity, even more preferably at least about 95% amino acid sequence identity and yet more preferably 98% amino acid sequence identity with the amino acid sequence of FIG. 2A (SEQ ID NO:3).

“Percent (c) amino acid sequence identity” with respect to the PRO364 amino acid sequences identified herein is defined as the percentage of amino acid residues in a candidate sequence that are identical with the amino acid residues in a PRO364 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Percent (%) nucleic acid sequence identity” with respect to the PRO364 sequence identified herein is defined as the percentage of nucleotides in a candidate sequence that are identical with the nucleotides in the PRO364 sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

The term “epitope tagged” where used herein refers to a chimeric polypeptide comprising a PRO364 polypeptide, or domain sequence thereof, fused to a “tag polypeptide”. The tag polypeptide has enough residues to provide an epitope against which an antibody may be made, or which can be identified by some other agent, yet is short enough such that it does not interfere with the activity of the PRO364 polypeptide. The tag polypeptide preferably is also fairly unique so that the antibody does not substantially cross-react with other epitopes. Suitable tag polypeptides generally have at least six amino acid residues and usually between about 8 to about 50 amino acid residues (preferably, between about 10 to about 20 residues).

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO364 polypeptide natural environment will not be present ordinarily, however, isolated polypeptide will be prepared by at least one purification step.

An “isolated” PRO364 polypeptide-encoding nucleic acid molecule is a nucleic acid molecule that is identified and separated from at least one contaminant nucleic acid molecule with which it is ordinarily associated in the natural source of the PRO364 polypeptide-encoding nucleic acid. An isolated PRO364 polypeptide-encoding nucleic acid molecule is other than in the form or setting in which it is found in nature. Isolated PRO364 polypeptide-encoding nucleic acid molecules therefore are distinguished from the PRO364 polypeptide-encoding nucleic acid molecule as it exists in natural cells. However, an isolated PRO364 polypeptide-encoding nucleic acid molecule includes PRO364 polypeptide-encoding nucleic acid molecules contained in cells that ordinarily express PRO364 polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers single anti-PRO364 polypeptide monoclonal antibodies (including agonist, antagonist, and neutralizing antibodies) and anti-PRO364 antibody compositions with polyepitopic specificity. The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Active” or “activity” for the purposes herein refers to form(s) of PRO364 which retain the biologic and/or immunologic activities of native or naturally-occurring PRO364 polypeptide. Such activities include, for instance, the ability to modulate (either in an agonistic or antagonistic manner) apoptosis, proinflammatory or autoimmune responses in mammalian cells. Agonistic activity will include the ability to stimulate or enhance an activity, while antagonistic activity will include the ability to block, suppress or neutralize an activity.

The terms “treating”, “treatment” and “therapy” as used herein refer to curative therapy, prophylactic therapy, and preventative therapy.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured, for instance, by cell viability assays, FACS analysis, or DNA electrophoresis, all which are known in the art.

The terms “cancer”, “cancerous”, and “malignant” refer to or describe the physiological condition in mammals that is typically characterized by unregulated cell growth. Examples of cancer include but are not limited to, carcinoma, including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

The term “mammal” as used herein refers to any mammal classified as a mammal, including humans, cows, horses, dogs and cats. In a preferred embodiment of the invention, the mammal is a human.

II. Compositions and Methods of the Invention

A. Full-Length PRO364 Polypeptitde

The present invention provides newly identified and isolated nucleotide sequences encoding polypeptides referred to in the present application as PRO364. In particular, Applicants have identified and isolated cDNA encoding a PRO364 polypeptide, as disclosed in further detail in the Examples below. Using BLAST and FastA sequence alignment computer programs (with set default parameters), Applicants found that portions of the PRO364 polypeptide have certain sequence identity with various members of the tumor necrosis factor receptor family. Accordingly, it is presently believed that PRO364 polypeptide disclosed in the present application is a newly identified member of the tumor necrosis factor receptor family of polypeptides.

It is believed that the PRO364 receptor is a human ortholog of the murine GITR. Relatively low levels of PRO364 mRNA expression were observed, and mainly in lymphoid tissues. However, peripheral blood T cells expressed abundant PRO364 upon stimulation, which suggests that the PRO364 receptor plays a role in T cell function. As shown in the Examples below, it is believed that the polypeptide encoded by the DNA19355-1150 nucleotide sequence may be a ligand for the PRO364 polypeptide receptor. Co-transfection of the PRO364 receptor and the DNA19355 ligand was found to protect human Jurkat T cells against AICD. These results suggest that the PRO364 receptor and ligand may modulate T lymphocyte survival in peripheral tissues and proinflammatory responses in mammals. The activation of NF-KB by the DNA19355 ligand/PRO364 interaction also suggests its role in modulating apoptosis, proinflammatory and autoimmune responses in mammalian cells. It is contemplated for instance, that a PRO364 immunoadhesin molecule (e.g., a PRO364 ECD-Ig construct) could be used in an antagonistic manner to block NF-κB activation by the DNA19355 ligand.

B. PRO164 Variants

In addition to the full-length native sequence PRO364 polypeptide described herein, it is contemplated that PRO364 variants can be prepared. PRO364 variants can be prepared by introducing appropriate nucleotide changes into the PRO364-encoding DNA, or by synthesis of the desired PRO364 polypeptide. Those skilled in the art will appreciate that amino acid changes may alter post-translational processes of the PRO364 polypeptide, such as changing the number or position of glycosylation sites or altering the membrane anchoring characteristics.

Variations in the native full-length sequence PRO364 or in various domains of the PRO364 polypeptide described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO364 polypeptide that results in a change in the amino acid sequence of the PRO364 polypeptide as compared with the native sequence PRO364. Optionally the variation is by substitution of at least one amino acid with any other amino acid in one or more of the domains of the PRO364 polypeptide. Guidance in determining which amino acid residue may be inserted, substituted or deleted without adversely affecting the desired activity may be found by comparing the sequence of the PRO364 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting variants for activity in any of the in vitro assays described in the Examples below.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans R. Soc. London SerA, 317:415 (1986)] or other known techniques can be performed on the cloned DNA to produce the PRO364-encoding variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of PRO194

Covalent modifications of PRO364 polypeptides are included within the scope of this invention. One type of covalent modification includes reacting targeted amino acid residues of a PRO364 polypeptide with an organic derivatizing agent that is capable of reacting with selected side chains or the N- or C-terminal residues of a PRO364 polypeptide. Derivatization with bifunctional agents is useful, for instance, for crosslinking PRO364 to a water-insoluble support matrix or surface for use in the method for purifying anti-PRO364 antibodies, and vice-versa. Commonly used crosslinking agents include, e.g., 1,1-bis(diazo-acetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO364 polypeptide included within the scope of this invention comprises altering the native glycosylation pattern of the polypeptide. “Altering the native glycosylation pattern” is intended for purposes herein to mean deleting one or more carbohydrate moieties found in native sequence PRO364 polypeptide, and/or adding one or more glycosylation sites that are not present in the native sequence PRO364 polypeptide.

Addition of glycosylation sites to PRO364 polypeptides may be accomplished by altering the amino acid sequence thereof. The alteration may be made, for example, by the addition of, or substitution by, one or more serine or threonine residues to the native sequence PRO364 polypeptide (for O-linked glycosylation sites). The PRO364 amino acid sequence may optionally be altered through changes at the DNA level, particularly by mutating the DNA encoding the PRO364 polypeptide at preselected bases such that codons are generated that will translate into the desired amino acids.

Another means of increasing the number of carbohydrate moieties on the PRO364 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO364 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO364 comprises linking the PRO364 polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

PRO364 polypeptides of the present invention may also be modified in a way to form chimeric molecules comprising a PRO364 polypeptide fused to another, heterologous polypeptide or amino acid sequence. In one embodiment, such a chimeric molecule comprises a fusion of a PRO364 polypeptide with a tag polypeptide which provides an epitope to which an anti-tag antibody can selectively bind. The epitope tag is generally placed at the amino- or carboxyl-terminus of the PRO364 polypeptide. The presence of such epitope-tagged forms of a PRO364 polypeptide can be detected using an antibody against the tag polypeptide. Also, provision of the epitope tag enables the PRO364 polypeptide to be readily purified by affinity purification using an anti-tag antibody or another type of affinity matrix that binds to the epitope tag. In an alternative embodiment, the chimeric molecule may comprise a fusion of a PRO364 polypeptide with an immunoglobulin or a particular region of an immunoglobulin. For a bivalent form of the chimeric molecule, such a fusion could be to the Fc region of an IgG molecule. Optionally, the chimeric molecule will comprise a PRO364 ECD sequence fused to an Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, (6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag (Lutz-Freyermuth et al., Proc. Natl. Acad. Sci. USA, 87:6393-6397 (1990)].

The PRO364 polypeptide of the present invention may also be modified in a way to form a chimeric molecule comprising a PRO364 polypeptide fused to a leucine zipper. Various leucine zipper polypeptides have been described in the art. See, e.g., Landschulz et al., Science 240:1759 (1988); WO 94/10308; Hoppe et al., FEBS Letters 344:1991 (1994); Maniatis et al., Nature 341:24 (1989). It is believed that use of a leucine zipper fused to a PRO364 polypeptide may be desirable to assist in dimerizing or trimerizing soluble PRO364 polypeptide in solution. Those skilled in the art will appreciate that the leucine zipper may be fused at either the N- or C-terminal end of the PRO364 molecule.

D. Preparation of PRO394

The description below relates primarily to production of PRO364 by culturing cells transformed or transfected with a vector containing PRO364 polypeptide encoding nucleic acid. It is, of course, contemplated that alternative methods, which are well known in the art, may be employed to prepare PRO364 polypeptides. For instance, the PRO364 sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of PRO364 polypeptides may be chemically synthesized separately and combined using chemical or enzymatic methods to produce a full-length PRO364 polypeptide.

1. Isolation of DNA Encoding PRO364

DNA encoding a PRO364 polypeptide may be obtained from a cDNA library prepared from tissue believed to possess the PRO364 mRNA and to express it at a detectable level. Accordingly, human PRO364-encoding DNA can be conveniently obtained from a cDNA library prepared from human tissue, such as described in the Examples. The PRO364-encoding gene may also be obtained from a genomic library or by oligonucleotide synthesis.

Libraries can be screened with probes (such as antibodies to a PRO364 polypeptide or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO364 is to use PCR methodology [Sambrook et al., supra; Dieffenbach et al., PCR Primer: A Laboratory Manual (Cold Spring Harbor Laboratory Press, 1995)]

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs such as ALIGN, DNAstar, and INHERIT.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### 2. Selection and Transformation of Host Cell

Host cells are transfected or transformed with expression or cloning vectors described herein for PRO364 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of transfection are known to the ordinarily skilled artisan, for example, CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic microbes such as filamentous fungi or yeast are suitable cloning or expression hosts for PRO364-encoding vectors. Saccharomyces cerevisiae is a commonly used lower eukaryotic host microorganism.

Suitable host cells for the expression of glycosylated PRO364 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen. Virol., 36:59 (1977)); Chinese hamster ovary cells/-DHFR(CHO, Urlaub and Chasin, Proc. Natl. Acad Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

### 3. Selection and Use of a Replicable Vector

The nucleic acid (e.g., cDNA or genomic DNA) encoding the desired PRO364 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication one or more marker genes, an enhancer element, a promoter, and a transcription termination sequence. Construction of suitable vectors containing one or more of these components employs standard ligation techniques which are known to the skilled artisan.

The desired PRO364 polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO364-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 21 plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO364-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci USA, 77:4216 (1980). A suitable selection gene for use in yeast is the trpl gene present in the yeast plasmid YRp7 [Stinchcomb et al., Nature, 282:39 (1979); Kingsman et al., Gene, 7:141 (1979); Tschemper et al., Gene, 10:157 (1980)). The trpl gene provides a selection marker for a mutant strain of yeast lacking the ability to grow in tryptophan, for example, ATCC No. 44076 or PEP4-1 (Jones, Genetics, 85:12 (1977)].

Expression and cloning vectors usually contain a promoter operably linked to the PRO364-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776), and hybrid promoters such as the tac promoter [deBoer et al., Proc. Natl. Acad., USA, 80:21-25 (1983)]. Promoters for use in bacterial systems also will contain a Shine-Dalgarno (S.D.) sequence operably linked to the DNA encoding the PRO364 polypeptide.

Examples of suitable promoting sequences for use with yeast hosts include the promoters for 3-phosphoglycerate kinase (Hitzeman et al., J. Biol. Chem., 25:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO364 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding a PRO364 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO364 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO364.

Still other methods, vectors, and host cells suitable for adaptation to the synthesis of PRO364 polypeptides in recombinant vertebrate cell culture are described in Gething et al., Nature, 239:620-625 (1981); Mantei et al., Nature, 281:40-46 (1979); EP 117,060; and EP 117,058.

### 4. Detecting (Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO364 polypeptide or against a synthetic peptide based on the DNA sequences provided herein or against exogenous sequence fused to PRO364-encoding DNA and encoding a specific antibody epitope.

5 Purification of Polypeptide

Forms of PRO364 may be recovered from culture medium or from host cell lysates. If membrane-bound, it can be released from the membrane using a suitable detergent solution (e.g. Triton-X 100) or by enzymatic cleavage. Cells employed in expression of PRO364 polypeptides can be disrupted by various physical or chemical means, such as freeze-thaw cycling, sonication, mechanical disruption, or cell lysing agents.

It may be desired to purify PRO364 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO364 polypeptide. Various methods of protein purification may be employed and such methods are known in the art and described for example in Deutscher, Methods in Enzymology, 182 (1990); Scopes, Protein Purification Principles and Practice, Springer-Verlag, New York (1982). The purification step(s) selected will depend, for example, on the nature of the production process used and the particular PRO364 polypeptide produced.

### E. Uses for PRO364

Nucleotide sequences (or their complement) encoding PRO364 polypeptides have various applications in the art of molecular biology, including uses as hybridization probes, in chromosome and gene mapping and in the generation of anti-sense RNA and DNA. PRO364-encoding nucleic acid will also be useful for the preparation of PRO364 polypeptides by the recombinant techniques described herein.

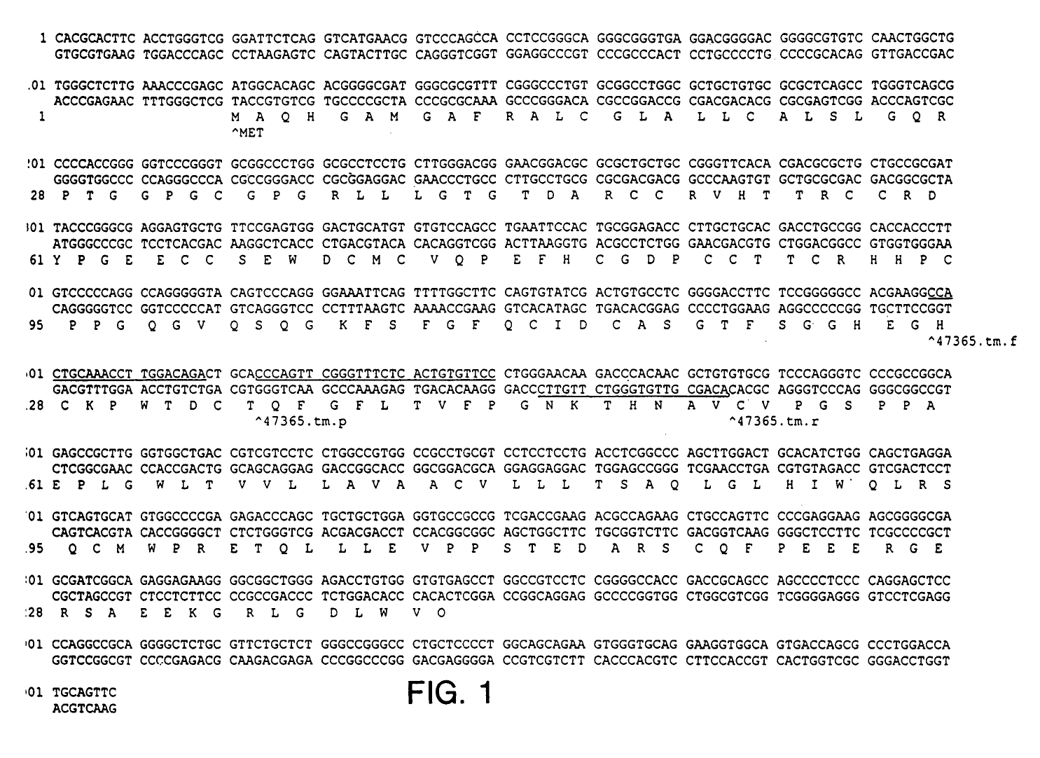

The full-length DNA47365-1206 nucleotide sequence (SEQ ID NO:1) or the full-length native sequence PRO364 (SEQ ID NO:2) nucleotide sequence, or portions thereof, may be used as hybridization probes for a cDNA library to isolate the full-length PRO364 gene or to isolate still other genes (for instance, those encoding naturally-occurring variants of PRO364 or PRO364 from other species) which have a desired sequence identity to the PRO364 nucleotide sequence disclosed in FIG. 1 (SEQ ID NO:1). Optionally, the length of the probes will be about 20 to about 50 bases. The hybridization probes may be derived from the UNQ319 (DNA47365-1206) nucleotide sequence of SEQ ID NO:1 as shown in FIG. 1 or from genomic sequences including promoters, enhancer elements and introns of native sequence PRO364-encoding DNA. By way of example, a screening method will comprise isolating the coding region of the PRO364 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the PRO364 gene of the present invention can be used to screen libraries of human cDNA, genomic DNA or mRNA to determine which members of such libraries the probe hybridizes to. Hybridization techniques are described in further detail in the Examples below.

The probes may also be employed in PCR techniques to generate a pool of sequences for identification of closely related PRO364 sequences.

Nucleotide sequences encoding a PRO364 polypeptide can also be used to construct hybridization probes for mapping the gene which encodes that PRO364 polypeptide and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for PRO364 encode a protein which binds to another protein (example, where the PRO364 polypeptide functions as a receptor), the PRO364 polypeptide can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor PRO364 polypeptide can be used to isolate other correlative ligand(s) apart from the ligand described in Example 2 below. Screening assays can be designed to find lead compounds that mimic the biological activity of a native PRO364 or a receptor for PRO364. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode PRO364 polypeptide or any of its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding PRO364 polypeptide can be used to clone genomic DNA encoding PRO364 in accordance with established techniques and the genomic sequences used to generate transgenic animals that contain cells which express DNA encoding PRO364. Methods for generating transgenic animals, particularly animals such as mice or rats, have become conventional in the art and are described, for example, in U.S. Pat. Nos. 4,736,866 and 4,870,009. Typically, particular cells would be targeted for PRO364 transgene incorporation with tissue-specific enhancers. Transgenic animals that include a copy of a transgene encoding PRO364 introduced into the germ line of the animal at an embryonic stage can be used to examine the effect of increased expression of DNA encoding PRO364. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of PRO364 can be used to construct a PRO364 “knock out” animal which has a defective or altered gene encoding PRO364 as a result of homologous recombination between the endogenous gene encoding PRO364 and altered genomic DNA encoding PRO364 introduced into an embryonic cell of the animal. For example, cDNA encoding PRO364 can be used to clone genomic DNA encoding PRO364 in accordance with established techniques. A portion of the genomic DNA encoding PRO364 can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)). The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the PRO364 polypeptide.

The PRO364 polypeptide herein may be employed in accordance with the present invention by expression of such polypeptides in vivo, which is often referred to as gene therapy.

There are two major approaches to getting the nucleic acid (optionally contained in a vector) into the patient's cells: in vivo and ex vivo. For in vivo delivery the nucleic acid is injected directly into the patient, usually at the sites where the PRO364 polypeptide is required, i.e., the site of synthesis of the PRO364 polypeptide, if known, and the site where biological activity of PRO364 polypeptide is needed. For ex vivo treatment, the patient's cells are removed, the nucleic acid is introduced into these isolated cells, and the modified cells are administered to the patient either directly or, for example, encapsulated within porous membranes that are implanted into the patient (see, e.g., U.S. Pat. Nos. 4,892,538 and 5,283,187).

There are a variety of techniques available for introducing nucleic acids into viable cells. The techniques vary depending upon whether the nucleic acid is transferred into cultured cells in vitro, or transferred in vivo in the cells of the intended host. Techniques suitable for the transfer of nucleic acid into mammalian cells in vitro include the use of liposomes, electroporation, microinjection, transduction, cell fusion, DEAE-dextran, the calcium phosphate precipitation method, etc. Transduction involves the association of a replication-defective, recombinant viral (preferably retroviral) particle with a cellular receptor, followed by introduction of the nucleic acids contained by the particle into the cell. A commonly used vector for ex vivo delivery of the gene is a retrovirus.

The currently preferred in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinson et al., Cancer Investigation, 14(1): 54-65 (1996)). The most preferred vectors for use in gene therapy are viruses, most preferably adenoviruses, AAV, lentiviruses, or retroviruses. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding PRO364 polypeptide, is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of the PRO364 polypeptide from a host cell in which it is placed. Preferably the signal sequence for this purpose is a mammalian signal sequence, most preferably the native signal sequence for PRO364 polypeptide. Optionally, the vector construct may also include a signal that directs polyadenylation, as well as one or more restriction sites and a translation termination sequence. By way of example, such vectors will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

In some situations, it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis may be used for targeting and/or to facilitate uptake, e.g,. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262:4429-4432 (1987); and Wagner et al., Proc., Natl. Acad. Sci. USA, 87:3410-3414 (1990). For a review of the currently known gene marking and gene therapy protocols, see Anderson et al., Science, 256:808-813 (1992). See also WO 93/25673 and the references cited therein.

Suitable gene therapy and methods for making retroviral particles and structural proteins can be found in, e.g., U.S. Pat. No. 5,681,746.

PRO364 polypeptides of the present invention which possess biological activity, for example such as related to that of the known tumor necrosis factor receptors may be employed both in vivo for therapeutic purposes and in vitro.

Therapeutic compositions of the PRO364 can be prepared by mixing the desired molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations or aqueous solutions. Acceptable carriers, excipients, or stabilizers are preferably nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturate-vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The PRO364 polypeptides will typically be formulated in such vehicles at a concentration of about 0.1 mg/ml to 100 mg/ml.

PRO364 polypeptide to be used for in vivo administration should be sterile. This is readily accomplished by filtration through sterile filtration membranes, prior to or following lyophilization and reconstitution. PRO364 polypeptide ordinarily will be stored in lyophilized form or in solution if administered systemically. If in lyophilized form, PRO364 polypeptide is typically formulated in combination with other ingredients for reconstitution with an appropriate diluent at the time for use. An example of a liquid formulation of PRO364 polypeptide is a sterile, clear, colorless unpreserved solution filled in a single-dose vial for subcutaneous injection.

Therapeutic PRO364 polypeptide compositions generally are placed into a container having a sterile access port, for example, an intravenous solution bag or vial having a stopper pierceable by a hypodermic injection needle. The formulations are preferably administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956).

PRO364 polypeptide can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed Mater. Res., 15:167-277 (1981) and Langer, Chem. Tech., 12:98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22:547-556 (1983)), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid (EP 133,988).

The therapeutically effective dose of a PRO364 polypeptide (or antibody thereto) will, of course, vary depending on such factors as the intended therapy (e.g., for modulating apoptosis, autoimmune or proinflammatory responses), the pathological condition to be treated, the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect.

With the above guidelines, the effective dose generally is within the range of from about 0.001 to about 1.0 mg/kg.

The route of PRO364 polypeptide administration is in accord with known methods, e.g., by injection or infusion by intravenous, intramuscular, intracerebral, intraperitoneal, intracerobrospinal, subcutaneous, intraocular, intraarticular, intrasynovial, intrathecal, oral, topical, or inhalation routes, or by sustained-release systems. The PRO364 also are suitably administered by intratumoral, peritumoral, intralesional, or perilesional routes, to exert local as well as systemic therapeutic effects.

The effectiveness of the PRO364 polypeptide treating the disorder may be improved by administering the active agent serially or in combination with another agent that is effective for those purposes, either in the same composition or as separate compositions.

Examples of such agents include cytotoxic, chemotherapeutic or growth-inhibitory agents, and radiological treatments (such as involving irradiation or administration of radiological substances).

The effective amounts of the therapeutic agents administered in combination with PRO364 polypeptide will be at the physician's discretion. Dosage administration and adjustment is done to achieve maximal management of the conditions to be treated.

F. Anti-PRPO34 Antibodies

The present invention further provides anti-PRO364 polypeptide antibodies. Exemplary antibodies include polyclonal, monoclonal, humanized, bispecific, and heteroconjugate antibodies.

1. Polyclonal Antibodies

The anti-PRO364 antibodies of the present invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO364 polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-PRO364 antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the PRO364 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against a PRO364 polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Humanized Antibodies

The anti-PRO364 antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 322:323-329 (1988); and Presta, Curr Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)).

4. Bispecific Antibodies

Bispecific antibodies are monoclonal, preferably human or humanized, antibodies that have binding specificities for at least two different antigens. In the present case, one of the binding specificities is for a PRO364 polypeptide, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 1:210 (1986).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

G. Uses for Anti-PRO364 Antibodies

The anti-PRO364 antibodies of the present invention have various utilities. The anti-PRO364 antibodies may be used in therapy, using techniques and methods of administration described above. Also, for example, anti-PRO364 antibodies may be used in diagnostic assays for PRO364 polypeptides, e.g., detecting expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 11:1014 (1974); Pain et al., J. Immunol. Meth., 40:219 (1981); and Nygren, J. Histochem and Cytochem., 30:407 (1982).

Anti-PRO364 antibodies also are useful for the affinity purification of PRO364 polypeptides from recombinant cell culture or natural sources. In this process, the antibodies against a PRO364 polypeptide are immobilized on a suitable support, such a Sephadex resin or filter paper, using methods well known in the art. The immobilized antibody then is contacted with a sample containing the PRO364 polypeptide to be purified, and thereafter the support is washed with a suitable solvent that will remove substantially all the material in the sample except the PRO364 polypeptide, which is bound to the immobilized antibody. Finally, the support is washed with another suitable solvent that will release the PRO364 polypeptide from the antibody.

H. Articles of Manufacture

An article of manufacture such as a kit containing PRO364 polypeptide or antibodies thereof useful for the diagnosis or treatment of the disorders described herein comprises at least a container and a label. Suitable containers include, for example, bottles, vials, syringes, and test tubes. The containers may be formed from a variety of materials such as glass or plastic. The container holds a composition that is effective for diagnosing or treating the condition and may have a sterile access port (for example, the container may be an intravenous solution bag or a vial having a stopper pierceable by a hypodermic injection needle). The active agent in the composition is the PRO364 or an antibody thereto. The label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a second or third container with another active agent as described above.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Isolation of cDNA Clones Encoding Human PRO364

An expressed sequence tag (EST) DNA database (LIFESEQ™, Incyte Pharmaceuticals, Palo Alto, Calif.) was searched and an EST (Incyte EST No. 3003460) was identified that showed homology to members of the tumor necrosis factor receptor (TNFR) family of polypeptides.

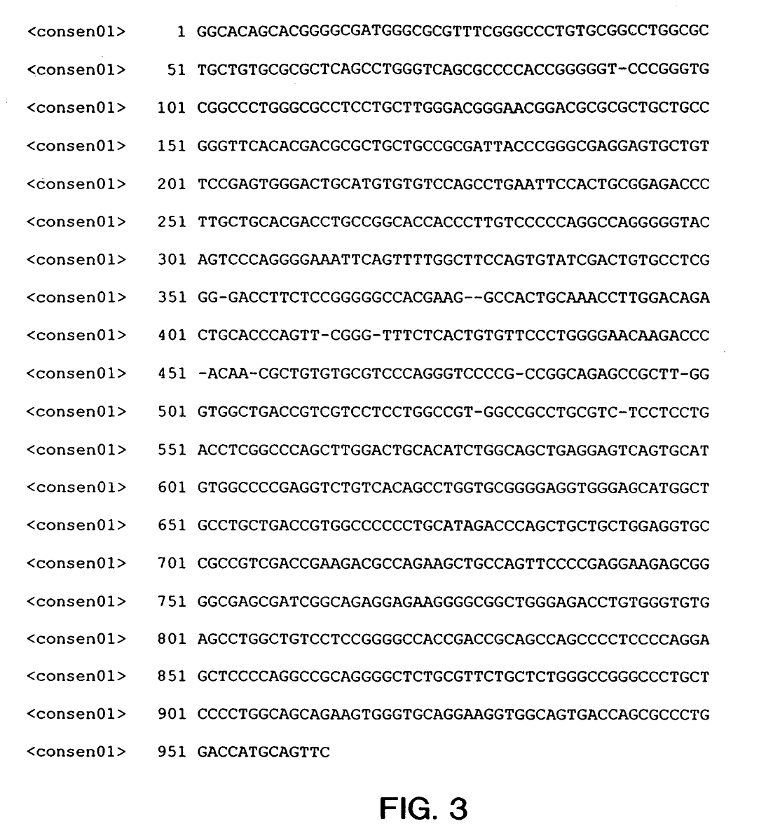

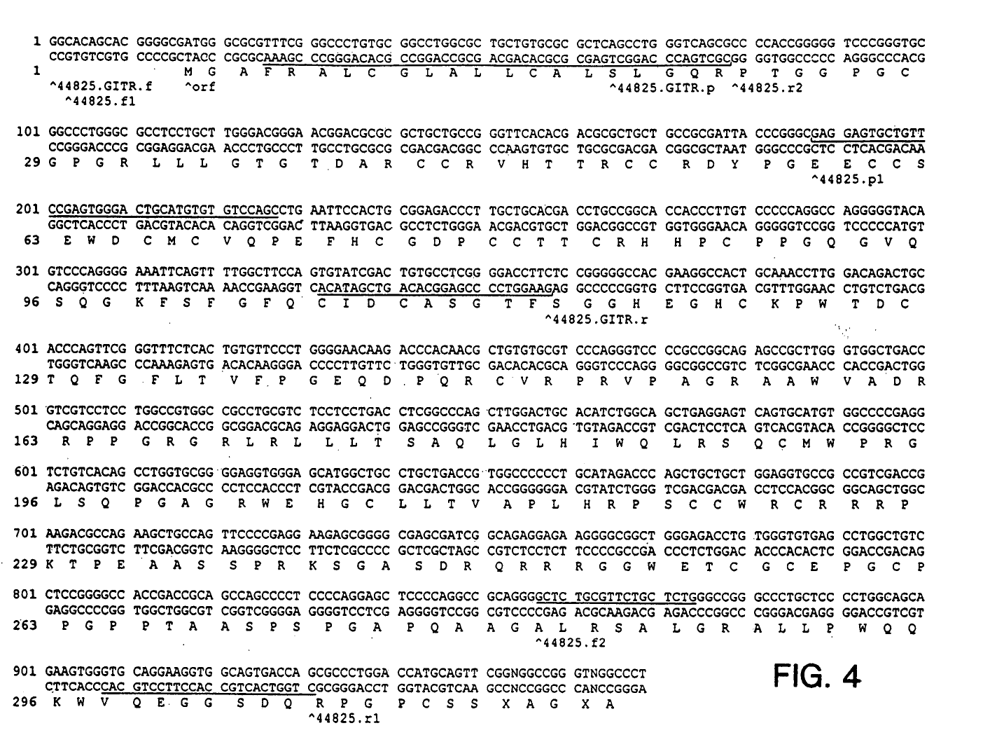

A consensus DNA sequence was then assembled relative to the Incyte 3003460 EST and other EST sequences using repeated cycles of BLAST (Altshul et al., Methods in Enzymology 266:460-480 (1996)) and “phrap” (Phil Green, University of Washington, Seattle, http://bozeman.mbt.washington.edu/phrap.docs/phrap.html). This consensus sequence is herein designated “<consen01>” in FIGS. 3A-C. The “<consen01>” consensus sequence shown in FIGS. 3A-C is also herein designated as “DNA44825” (see FIG. 4).

Based upon the DNA44825 and “<consen1>” consensus sequences shown in FIGS. 3-4, oligonucleotides were synthesized: 1) to identify by PCR a cDNA library that contained the sequence of interest, and 2) for use as probes to isolate a clone of the full-length coding sequence for PRO364. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was then used to isolate clones encoding the gene of interest using the probe oligonucleotide and one of the primer pairs.

Pairs of PCR primers (forward and reverse) were synthesized:

| | |
|---|---|
| forward PCR primer (44825.f1) | |
| 5′-CACAGCACGGGGCGATGGG-3′ | (SEQ ID NO:5) |
| forward PCR primer (44825.f2) | |
| 5′-GCTCTGCGTTCTGCTCTG-3′ | (SEQ ID NO:6) |
| forward PCR primer (44825.GITR.f) | |
| 5′-GGCACAGCACGGGGCGATGGGCGCGTTT-3′ | (SEQ ID NO:7) |
| reverse PCR primer (44825.r1) | |
| 5′-CTGGTCACTGCCACCTTCCTGCAC-3′ | (SEQ ID NO:8) |
| reverse PCR primer (44825.r2) | |
| 5′-CGCTGACCCAGGCTGAG-3′ | (SEQ ID NO:9) |
| reverse PCR primer (44825.GITR.r) | |
| 5′-GAAGGTCCCCGAGGCACAGTCGATACA-3′ | (SEQ ID NO:10) |

Additionally, synthetic oligonucleotide hybridization probes were constructed from the consensus DNA44825 sequence which had the following nucleotide sequences

(SEQ ID NO:11)

hybridization probe (44825.p1)
5′-GAGGAGTGCTGTTCCGAGTGGGACTGCATGTGTGTCCAGC-3′

(SEQ ID NO:12)

hybridization probe (44825.GITR.p)
5′-AGCCTGGGTCAGCGCCCCACCGGGGGTCCCGGGTGCGGCC-3′

In order to screen several libraries for a source of a full-length clone, DNA from the libraries was screened by PCR amplification with the PCR primer pairs identified above. A positive library was then used to isolate clones encoding the PRO364 gene using the probe oligonucleotides and one of the PCR primers.

RNA for construction of the cDNA libraries was isolated from human bone marrow tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991) in the unique XhoI and NotI sites.

DNA sequencing of the clones isolated as described above gave the full-length DNA sequence for PRO364 [herein designated as UNQ319 (DNA47365-1206)] (SEQ ID NO:1) and the derived protein sequence for PRO364.

The entire nucleotide sequence of UNQ319 (DNA47365-1206) is shown in FIG. 1 (SEQ ID NO:1). Clone UNQ319 (DNA47365-1206) has been deposited with ATCC and is assigned ATCC Deposit No. ATCC 209436. Clone UNQ319 (DNA47365-1206) contains a single open reading frame with an apparent translational initiation site at nucleotide positions 121-123 [Kozak et al., supra] and ending at the stop codon at nucleotide positions 844-846 (FIG. 1). The predicted polypeptide precursor is 241 amino acids long (FIG. 2A). The full-length PRO364 protein shown in FIG. 2A has an estimated molecular weight of about 26,000 daltons and a pI of about 6.34. A potential N-glycosylation site exists between amino acids 146 and 149 of the amino acid sequence shown in FIG. 2A. Hydropathy analysis (not shown) suggested a Type I transmembrane typology; a putative signal sequence is from amino acids 1 to 25 and a potential transmembrane domain exists between amino acids 162 to 180 of the sequence shown in FIG. 2A.

Analysis of the amino acid sequence of the full-length PRO364 polypeptide suggests that portions of it possess homology to members of the tumor necrosis factor receptor family, thereby indicating that PRO364 may be a novel member of the tumor necrosis factor receptor family. The intracellular domain of PRO364 contains a motif (in the region of amino acids 207-214 of FIG. 2A) similar to the minimal domain within the CD30 receptor shown to be required for TRAF2 binding and which is also present within TNFR2 [Lee et al., supra, (1996)). There are three apparent extracellular cysteine-rich domains characteristic of the TNFR family [see, Naismith and Sprang, Trends Biochem. Sci., 23:74-79 (1998)], of which the third CRD has 3 rather than the more typical 4 or 6 cysteines of the TNFR family. As compared to the mouse GITR (described below) the PRO364 amino acid sequence has 8 cysteines in CRD1 relative to 5 cysteines in CRD1 of mouse GITR, and the presence of one potential N-linked glycosylation site in the ECD as compared to 4 potential N-linked glycosylation sites in mouse GITR (see FIG. 2B).

A detailed review of the putative amino acid sequence of the full-length native PRO364 polypeptide and the nucleotide sequence that encodes it evidences sequence homology with the mouse GITR (mGITR) protein reported by Nocentini et al., Proc., Natl. Acad. Sci. USA 94:6216-6221 (1997). It is possible, therefore, that PRO364 represents the human counterpart or ortholog to the mouse GITR protein reported by Nocentini et al. A comparison of the PRO364 polypeptide and the mGITR amino acid sequences is shown in FIG. 2B.

### Example 2

### Identification of a Potential Ligand for the PRO164 Polypeptide

A cDNA clone that encodes a novel polypeptide which may be a ligand that binds to the PRO364 polypeptide described herein was isolated as follows. Methods described in Klein et al., Proc. Natl. Acad. Sci. USA 93:7108-7113 (1996) were employed with the following modifications. Yeast transformation was performed with limiting amounts of transforming DNA in order to reduce the number of multiple transformed yeast cells. Instead of plasmid isolation from the yeast followed by transformation of E. coli as described in Klein et al., supra, PCR analysis was performed on single yeast colonies. This was accomplished by restreaking the original sucrose positive colony onto fresh sucrose medium to purify the positive clone. A single purified colony was then used for PCR using the following primers: 5′-TGTAAAACGACGGCCAGTTTCTCTCAGAGAAACAAGCAAAAC-3′ (SEQ ID NO:13) and 5′-CAGGAAACAGCTATGACCGAAGTGGACCAAAGGTCTATCGCTA-3′ (SEQ ID NO:14). The PCR primers are bipartite in order to amplify the insert and a small portion of the invertase gene (allowing to determine that the insert was in frame with invertase) and to add on universal sequencing primer sites.

A library of cDNA fragments derived from human umbilical cord endothelial (HUVEC) cells fused to invertase was transformed into yeast and transformants were selected on SC-URA media. URA and transformants were replica plated onto sucrose medium in order to identify clones secreting invertase. Positive clones were re-tested and PCR products were sequenced. The sequence of one clone, DNA1840, was determined to contain a signal peptide coding sequence. Oligonucleotide primers and probes were designed using the nucleotide sequence of DNA1840. A full length plasmid library of cDNAs from human umbilical vein endothelial cells was titered and approximately 100,000 cfu were plated in 192 pools of 500 cfu/pool into 96-well round bottom plates. The pools were grown overnight at 37° C. with shaking (200 rpm). PCR was performed on the individual cultures using primers specific to DNA1840. Agarose gel electrophoresis was performed and positive wells were identified by visualization of a band of the expected size. Individual positive clones were obtained by colony lift followed by hybridization with ³²P-labeled oligonucleotide. These clones were characterized by PCR, restriction digest, and Southern blot analyses.

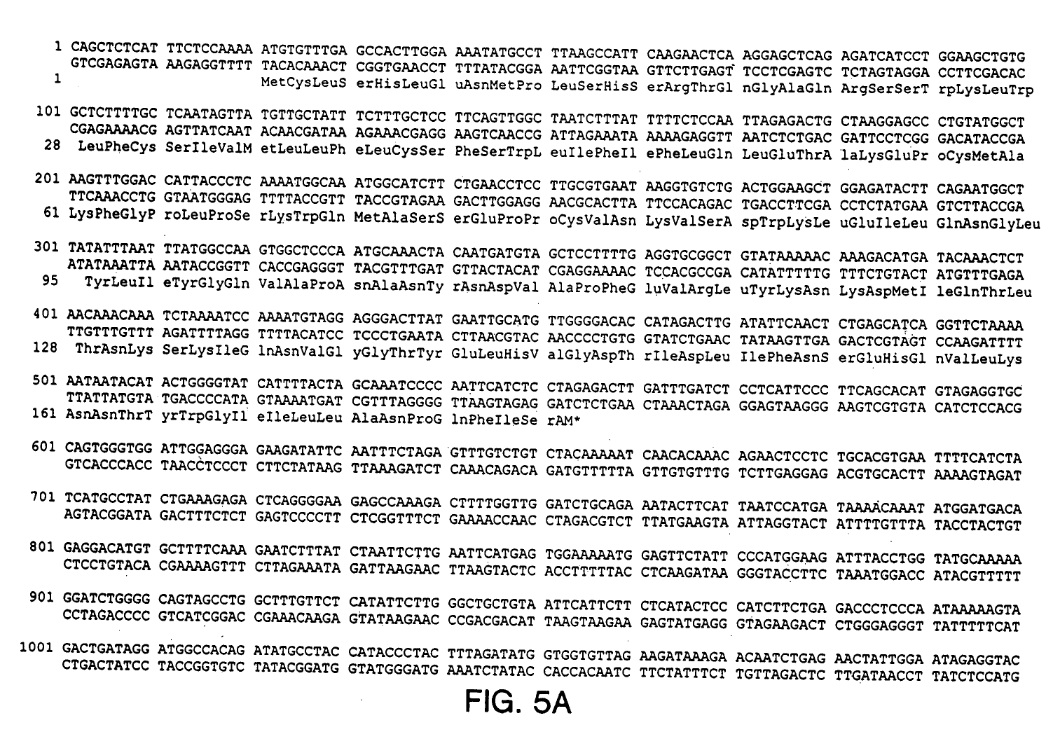

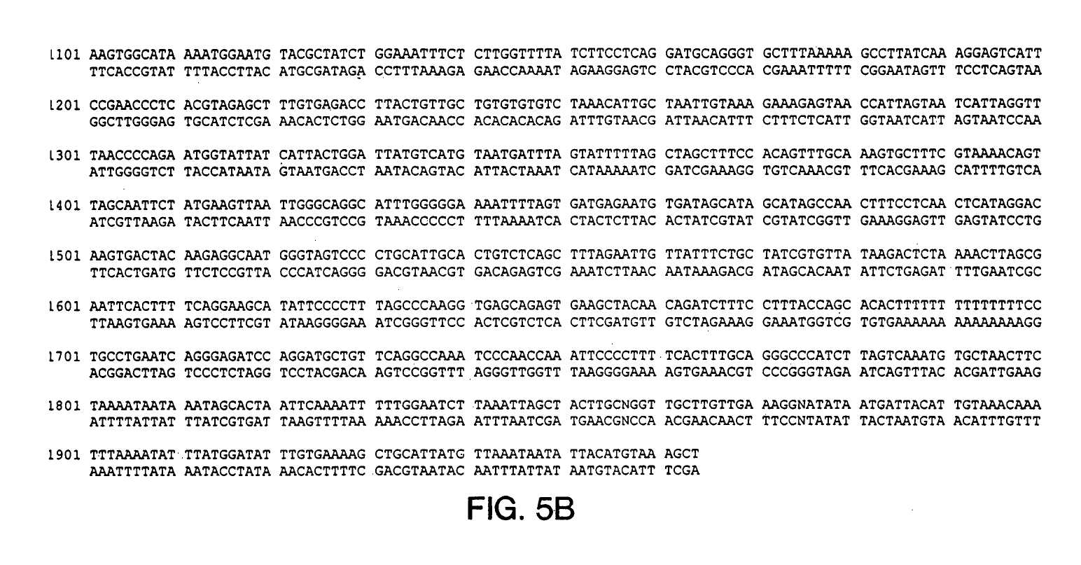

A cDNA clone was sequenced in entirety, wherein the complete sequence of the cDNA clone was designated DNA19355-1150. A nucleotide sequence of the DNA19355-1150 clone is shown in FIGS. 5A-B (SEQ ID NO:15). Clone DNA19355-1150 contains a single open reading frame with an apparent translational initiation site at nucleotide positions 21-23 [Kozak et al., supra] (FIGS. 5A-B). The predicted polypeptide precursor is 177 amino acids long (SEQ ID NO:16) and has a calculated molecular weight of approximately 20,308 daltons. Hydropathy analysis suggests a type II transmembrane protein typology, with a putative cytoplasmic region (amino acids 1-25); transmembrane region (amino acids 26-51); and extracellular region (amino acids 52-177). Two potential N-linked glycosylation sites have been identified at position 129 (Asn) and position 161 (Asn) of the sequence shown in FIGS. 5A-B (SEQ ID NO:15). Clone DNA19355-1150 has been deposited with ATCC on Nov. 18, 1997 and is assigned ATCC deposit no. 209466. The polypeptide encoded by DNA19355-1150 is obtained or obtainable by expressing the molecule encoded by the cDNA insert of the deposited ATCC 209466 vector. Digestion of the vector with XbaI and NotI restriction enzymes will yield a 1411 bp fragment and 668 bp fragment.

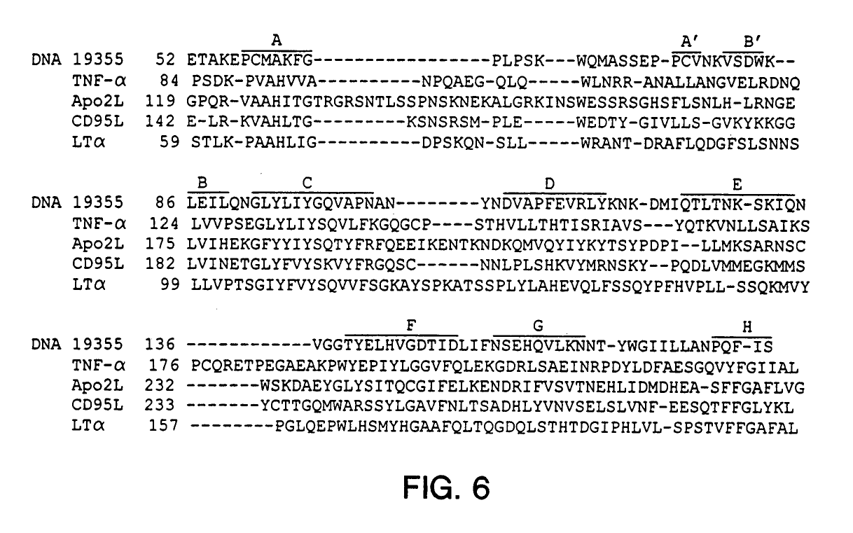

Based upon a BLAST and FastA sequence alignment analysis (using the ALIGN computer program) of extracellular sequence, DNA19355-1150 shows amino acid sequence identity to several members of the TNF cytokine family, and particularly, to human Apo-2L (19.8%), Fas/Apol-ligand (19.0%), TNF-alpha (20.6%) and Lymphotoxin-α (17.5%) (see FIG. 6).

Analysis of the polypeptide encoded by the DNA19355-1150 nucleotide sequence indicates that it is a potential ligand for the human PRO364 polypeptide described herein.

### Example 3

### Use of PRO364-Encoding DNA as a Hybridization Probe

The following method describes use of a nucleotide sequence encoding PRO364 as a hybridization probe.

DNA comprising the coding sequence of full-length PRO364 (as shown in FIG. 1, SEQ ID NO:1) or a fragment thereof is employed as a probe to screen for homologous DNAs (such as those encoding naturally-occurring variants of PRO364) in human tissue cDNA libraries or human tissue genomic libraries.

Hybridization and washing of filters containing either library DNAs is performed under the following high stringency conditions. Hybridization of radiolabeled PRO364 polypeptide-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PRO364 polypeptide can then be identified using standard techniques known in the art.

### EXAMPLE 4

### Expression of PRO364 Polypeptides in E. coli

This example illustrates the preparation of forms of PRO364 polypeptides by recombinant expression in E. coli.

The DNA sequence encoding the full-length PRO364 (SEQ ID NO:3) or a fragment or variant thereof is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO364 coding region, lambda transcriptional terminator, and an argu gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO364 polypeptide can then be purified using a metal chelating column under conditions that allow tight binding of the polypeptide.

### Example 5

### Expression of PRO364 Polypeptides in Mammalian Cells

This example illustrates preparation of forms of PRO364 polypeptides by recombinant expression in mammalian cells.

The vector, pRK5 (see EP 307,247, published Mar. 15, 1989), is employed as the expression vector. Optionally, the PRO364-encoding DNA is ligated into pRKS with selected restriction enzymes to allow insertion of the PRO364-encoding DNA using ligation methods such as described in Sambrook et al., supra The resulting vector is called pRK5-PRO364.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO364 DNA is mixed with about 1 μg DNA encoding the VA RNA gene (Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for, about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO364 polypeptide. The cultures containing transfected cells may undergo further incubation (in serum free medium) and the medium is tested in selected bioassays.

In an alternative technique, PRO364-encoding DNA may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO364 DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO364 polypeptide can then be concentrated and purified by any selected method, such as dialysis and/or column chromatography.

In another embodiment, PRO364 polypeptide can be expressed in CHO cells. The pRK5-PRO364 vector can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO364 polypeptide, the culture medium may be replaced with serum free medium. Preferably, the cultures are incubated for about 6 days, and then the conditioned medium is harvested. The medium containing the expressed PRO364 polypeptide can then be concentrated and purified by any selected method.

Epitope-tagged PRO364 polypeptide may also be expressed in host CHO cells. The PRO364-encoding DNA may be subcloned out of the pRK5 vector. The subclone insert can undergo PCR to fuse in frame with a selected epitope tag such as a poly-his tag into a Baculovirus expression vector. The poly-his tagged PRO364-encoding DNA insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged PRO364 polypeptide can then be concentrated and purified by any selected method, such as by Ni²′-chelate affinity chromatography.

### Example 6

### Expression of a PRO364 Polypeptide in Yeast

The following method describes recombinant expression of PRO364 polypeptides in yeast.

First, yeast expression vectors are constructed for intracellular production or secretion of PRO364 polypeptide from the ADH2/GAPDH promoter. DNA encoding the PRO364 polypeptide of interest, a selected signal peptide and the promoter is inserted into suitable restriction enzyme sites in the selected plasmid to direct intracellular expression of the PRO364 polypeptide. For secretion, DNA encoding the PRO364 polypeptide can be cloned into the selected plasmid, together with DNA encoding the ADH2/GAPDH promoter, the yeast alpha-factor secretory signal/leader sequence, and linker sequences (if needed) for expression of the PRO364 polypeptide.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO364 polypeptide can subsequently be isolated and purified by removing the yeast cells from the fermentation medium by centrifugation and then concentrating the medium using selected cartridge filters. The concentrate containing the PRO364 polypeptide may further be purified using selected column chromatography resins.

### EXAMPLE 7

### Expression of PRO364 Polypeptides in Baculovirus-Infected Insect Cells

The following method describes recombinant expression of PRO364 polypeptides in Baculovirus-infected insect cells.

The PRO364-encoding DNA is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the PRO364-encoding DNA or the desired portion of the PRO364-encoding DNA (such as the sequence encoding the extracellular domain of a transmembrane protein) is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4 to 5 days of incubation at 28° C., the released viruses are harvested and used for further amplifications. Viral infection and protein expression is performed as described by O'Reilley et al., Baculovirus expression vectors: A laboratory Manual, Oxford:Oxford University Press (1994).

Expressed poly-his tagged PRO364 polypeptide can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% Glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% Glycerol, pH 7.8) and filtered through a 0.45 Mm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% Glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or western blot with Ni²⁺-NTA-conjugated to alkaline phosphatase (Qiagen). Fractions containing the eluted His₁₀-tagged PRO364 polypeptide are pooled and dialyzed against loading buffer.

Alternatively, purification of the IgG tagged (or Fc tagged) PRO364 polypeptide can be performed using known chromatography techniques, including for instance, Protein A or protein G column chromatography.

### Example 8

### Preparation of Antibodies that Bind PRO164 Polypeptides

This example illustrates the preparation of monoclonal antibodies which can specifically bind to PRO364 polypeptides.

Techniques for producing the monoclonal antibodies are known in the art and are described, for instance, in Goding, supra. Immunogens that may be employed include purified PRO364 polypeptide, fusion proteins containing a PRO364 polypeptide, and cells expressing recombinant PRO364 polypeptide on the cell surface. Selection of the immunogen can be made by the skilled artisan without undue experimentation.

Mice, such as Balb/c, are immunized with the PRO364 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO364 polypeptide antibodies.

After a suitable antibody titer has been detected, the animals “positive” for antibodies can be injected with a final intravenous injection of PRO364 polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO364 polypeptide. Determination of “positive” hybridoma cells secreting the desired monoclonal antibodies against a PRO364 polypeptide is within the skill in the art.

The positive hybridoma cells can be injected intraperitoneally into syngeneic Balb/c mice to produce ascites containing the anti-PRO364 polypeptide monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 9

### Assays to Detent Expression of PRO364 mRNA in Human Cells and Tissues

Assays were conducted to examine expression of PRO364 mRNA in normal human tissues and in cancer cells lines.

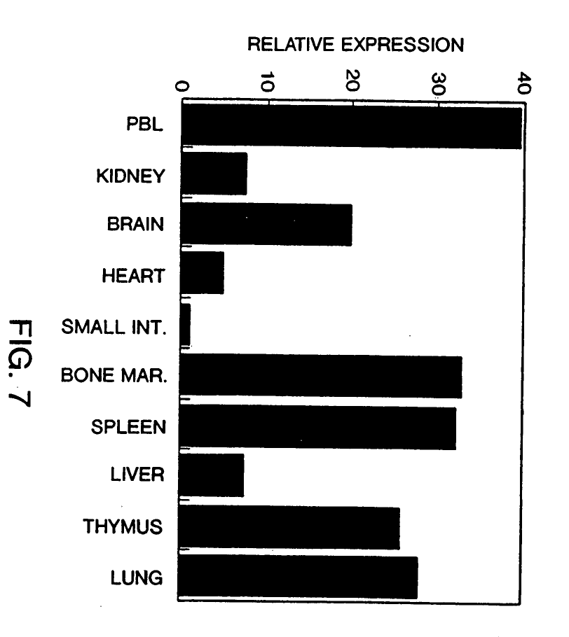

Various human tissues and cancer cell lines (Clontech) were tested by Northern blot hybridization for detection of PRO364 transcripts, but none were detected. Using quantitative reverse-transcriptase PCR, PRO364 mRNA was detected in PBL, brain, bone marrow, spleen, thymus and lung, and at relatively lower levels, in kidney, heart, small intestine and liver tissues (see FIG. 7). The relative mRNA expression levels were determined by quantitative PCR using a Taqman instrument (ABI) essentially as described in Heid et al., Genome Res., 6:986-94 (1996) using PRO364 specific primers and fluorogenic probes:

(SEQ ID NO:20)

DNA47365.tm.f - CCACTGAAACCTTGGACAGA

(SEQ ID NO:21)

DNA47365.tm.p - CCCAGTTCGGGTTTCTCACTGTGTTCC

(SEQ ID NO:22)

DNA47365.tm.r - ACAGCGTTGTGGGTCTTGTTC

The authenticity of the PCR product was confirmed by Southern blot hybridization to the corresponding cDNA. Expression levels were normalized relative to small intestine tissue.

In a separate assay, primary human T cells (isolated from donor whole blood using a T cell enrichment column (R & D Systems)) and monocytes/macrophages (isolated from donor whole blood by adherence to tissue culture flasks) were maintained in RPMI supplemented with 10% FBS and 2 mM glutamine. The cells were then treated for 24 hours with PHA (1 microgram/ml; Sigma), anti-CD3 antibody (1 microgram/ml; Pharmingen), LPS (1 microgram/ml; Sigma), TNF-alpha (1 microgram/ml; prepared essentially as described in Pennica et al., Nature, 312:724-729 (1984)), or the soluble DNA19355 ligand (5 microgram/ml; prepared as described in Example 10 below). The relative mRNA expression levels were then analyzed by the Taqman procedure described above. The expression levels were normalized relative to buffer-treated T cells.

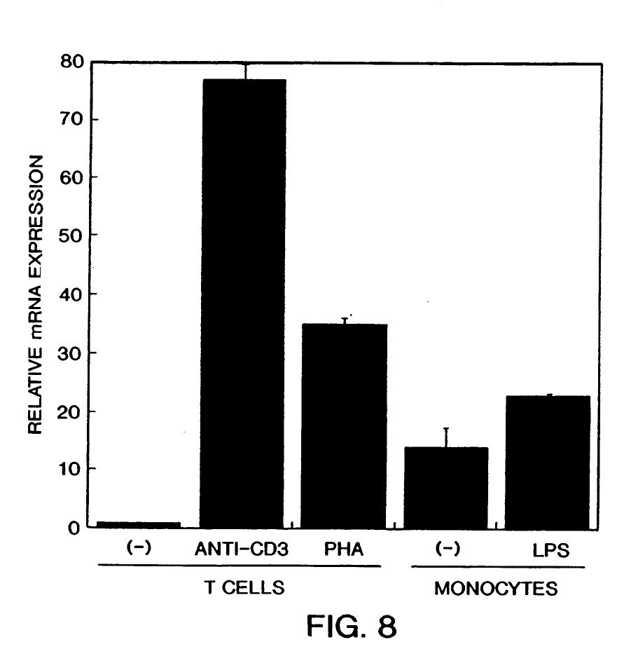

The results are shown in FIG. 8. Substantial up-regulation of PRO364 mRNA was observed in isolated peripheral blood T cells after stimulation by phytohemagglutinin (PHA) or by anti-CD3 antibody. High levels of expression were observed in isolated monocytes/macrophages and this expression was further increased by LPS. (See FIG. 8).

### Example 10

### Binding Specificity of DNA19355 for the PRO364 Receptor

Assays were conducted to determine whether the DNA19355 polypeptide (described in Example 2 above) interacts and specifically binds with PRO364, which is believed to be a human ortholog of the murine GITR (mGITR) polypeptide described in Nocentini et al., Proc. Natl. Acad. Sci., 94:6216-6221 (1997).

To test for binding, a soluble immunoglobulin fusion protein (immunoadhesin) which included a PRO364 extracellular domain (see amino acids 1-161 of FIG. 2A) was expressed in insect cells. The PRO364 ECD was expressed as a C-terminus IgG-Fc tagged form in insect cells using Baculovirus (as described in Example 7 above).

A soluble DNA19355 polypeptide was prepared by expressing an ECD in E. coli cells. The DNA sequence encoding an extracellular region of the DNA19355 polypeptide (amino acids 52 to 177 of FIG. 5A-B; SEQ ID NO:16) was amplified with PCR primers containing flanking NdeI and XbaI restriction sites, respectively: forward: 5′-GAC GAC AAG CAT ATG TTA GAG ACT GCT AAG GAG CCC TG-3′ (SEQ ID NO:17); reverse: 5′-TAG CAG CCG GAT CCT AGG AGA TGA ATT GGG GATT-3′ (SEQ ID NO:18). The PCR product was digested and cloned into the NdeI and XbaI sites of plasmid pET19B (Novagen) downstream and in frame of a Met Gly His10 sequence followed by a 12 amino acid enterokinase cleavage site (derived from the plasmid): Met Gly His His His His His His His His His His Ser Ser Gly His Ile Asp Asp Asp Asp Lys His Met (SEQ ID NO:19).

The resulting plasmid was used to transform E. Coli strain JM109 (ATCC 53323) using the methods described in Sambrook et al., supra. Transformants were identified by PCR. Plasmid DNA was isolated and confirmed by restriction analysis and DNA sequencing.

Selected clones were grown overnight in liquid culture medium LB supplemented with antibiotics. The overnight culture was subsequently used to inoculate a larger scale culture. The cells were grown to a desired optical density, during which the expression promoter is turned on.

After culturing the cells for several more hours, the cells were harvested by centrifugation. The cell pellet obtained by the centrifugation was solubilized using a microfluidizer in a buffer containing 0.1M Tris, 0.2M NaCl, 50 mM EDTA, pH 8.0. The solubilized DNA19355 protein was purified using Nickel-sepharose affinity chromatography.

The DNA19355 protein was analyzed by SDS-PAGE followed by Western blot with nickel-conjugated horseradish peroxidase followed by ECL detection (Boehringer Mannheim). Three predominant bands were detected, which corresponded in size to monomeric, homodimeric, and homotrimeric forms of the protein. It is believed based on this result that in its native form, in the absence of SDS denaturation, the soluble DNA19355 protein is capable of forming homotrimers.

The soluble DNA19355 ECD molecule was then labeled with ¹²⁵I, for testing its ability to interact with the PRO364 immunoadhesin. For comparison, immunoadhesin constructs were also made of the following TNF receptor family members: CD95, DR4, DR5, TNFR1, TNFR2, and Apo-3. CD95, DR4, DR5, TNFR1, TNFR2, and Apo-3 immunoadhesins were prepared by fusing each receptor's ECD to the hinge and Fc portion of human IgG, as described previously for TNFR1 [Ashkenazi et al., Proc. Natl. Acad. Sci., 88:10535-10539 (1991)]. The respective TNF receptor family members are described (and relevant references cited) in the Background of the Invention section.

For the co-precipitation assay, each immunoadhesin (5 microgram) was incubated with ¹²⁵I-labeled soluble DNA19355 polypeptide (1 microgram) for 1 hour at 24° C., followed by protein A-sepharose for 30 minutes on ice. The reaction mixtures were spun down and washed several times in PBS, boiled in SDS-PAGE buffer containing 20 mM dithiothreitol and then resolved by SDS-PAGE and autoradiography.

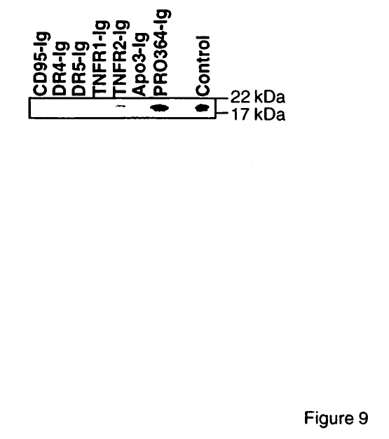

The results are shown in FIG. 9. The position of the molecular weight markers (kDa) are indicated in the figure. The PRO364-IgG bound to the radioiodinated soluble DNA19355 polypeptide. However, the PRO364-IgG did not bind to the immunoadhesin constructs of CD95, DR4, DR5, TNFR1, TNFR2, or Apo-3.

In another assay, human 293 cells were transiently transfected with full-length DNA19355 and the ability of receptor immunoadhesin constructs for PRO364, TNFR1, HVEM, and DcR1 to bind to those transfected cells was determined by FACS analysis. The 293 cells were maintained in high glucose DMEM media supplemented with 10% fetal bovine serum (FBS), 2 mM glutamine, 100 microgram/ml penicillin, and 100 microgram/ml streptomycin. The transfected cells (1×10⁵) were incubated for 60 minutes at 4° C. in 200 microliters 2% FBS/PBS with 1 microgram of the respective receptor or ligand immunoadhesin. The cells were then washed with 2% FBS/PBS, stained with R-phycoerythrin-conjugated goat anti-human antibody (Jackson Immunoresearch, West Grove, Pa.). Next, the cells were analyzed by FACS. To test the binding of the respective immunoadhesins to the transiently transfected cells, an expression vector (pRK5-CD4; Smith et al., Science, 328:1704-1707 (1987)) for CD4 was co-transfected with DNA19355 expression vector (see above). FITC-conjugated anti-CD4 (Pharmingen, San Diego, Calif.) was then used to identify and gate the transfected cell population in the FACS analysis.

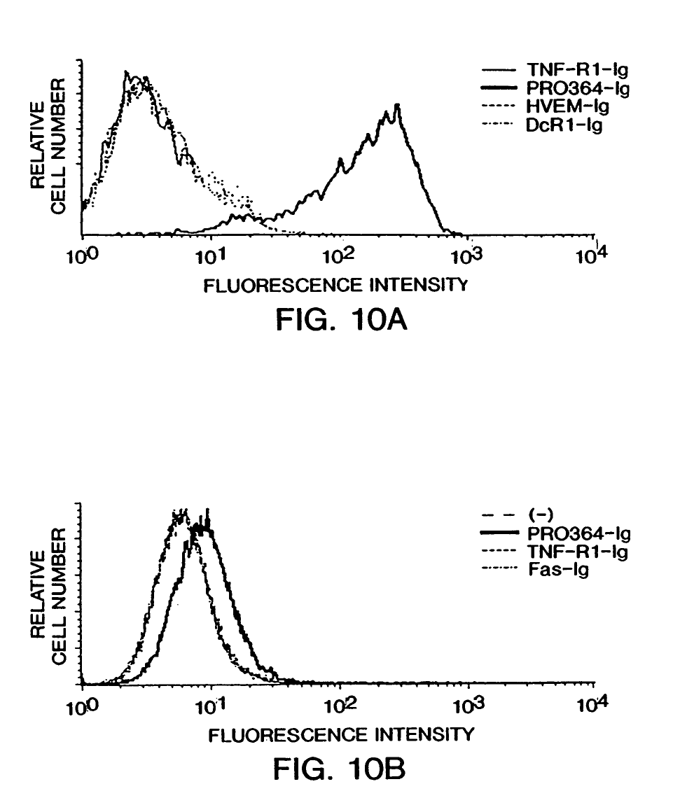

As shown in FIG. 10A, the PRO364-IgG bound specifically to the surface of cells transfected with the expression plasmid encoding the full length DNA19355. No such binding was observed for the TNFR1, HVEM or DcR1 immunoadhesins. The PRO364-IgG did not bind to the cells transfected with a control plasmid (data not shown).

The results demonstrate a specific binding interaction of the DNA19355 polypeptide with PRO364 and that the DNA19355 polypeptide does not interact with any of the other TNF receptor family members tested.

The DNA19355 polypeptide was identified in a human umbilical vein endothelial cell (HUVEC) library, and the DNA19355 polypeptide transcripts are readily detectable in HUVEC by RT-PCR (data not shown). A FACS analysis assay was conducted to examine whether specific binding of PRO364-IgG could be demonstrated with HUVEC by FACS analysis. HUVEC were purchased from Cell Systems (Kirkland, Wash.) and grown in a 50:50 mix of Ham's F12 and Low Glucose DMEM media containing 10 fetal bovine serum, 2 mM L-glutamine, 10 mM Hepes, and 10 ng/ml basic FGF. Cells were FACS sorted with PBS, PRO364-IgG, TNFR1-IgG or Fas-IgG as a primary antibody and goat anti-human F(ab′)₂ conjugated to phycoerythrin (CalTag, Burlingame, Calif.).

It was found that PRO364-IgG specifically bound to HUVEC. (See FIG. 10B). Neither the Fas-IgG nor the TNFR1-IgG exhibited specific binding to the endothelial cells.

### Example 11

### Activation of NP-κB by DNA19355

An assay was conducted to determine whether DNA19355/PRO364 induces NF-κB activation by analyzing expression of a reporter gene driven by a promoter containing a NF-κB responsive element from the E-selectin gene.

Human 293 cells (2×10⁵) (maintained in HG-DMEM supplemented with 10% FBS, 2 mM glutamine, 100 microgram/ml penicillin, and 100 microgram streptomycin) were transiently transfected by calcium phosphate transfection with 0.5 microgram of the firefly luciferase reporter plasmid pGL3.ELAM.tk [Yang et al., Nature, 395:284-288 (1998)] and 0.05 microgram of the Renilla luciferase reporter plasmid (as internal transfection control) (Pharmacia), as well as the indicated additional expression vectors for DNA19355 and PRO364 (described above) (0.1 microgram PRO364; 0.5 microgram for DNA19355 expression vector and other vectors referred to below), and carrier plasmid pRKSD to maintain constant DNA between transfections. After 24 hours, the transfected cells were harvested and luciferase activity was assayed as recommended by the manufacturer (Pharmacia). Activities (average of triplicate wells) were normalized for differences in transfection efficiency by dividing firefly luciferase activity by that of Renilla luciferase activity and were expressed as activity relative to that seen in the absence of added expression vectors.

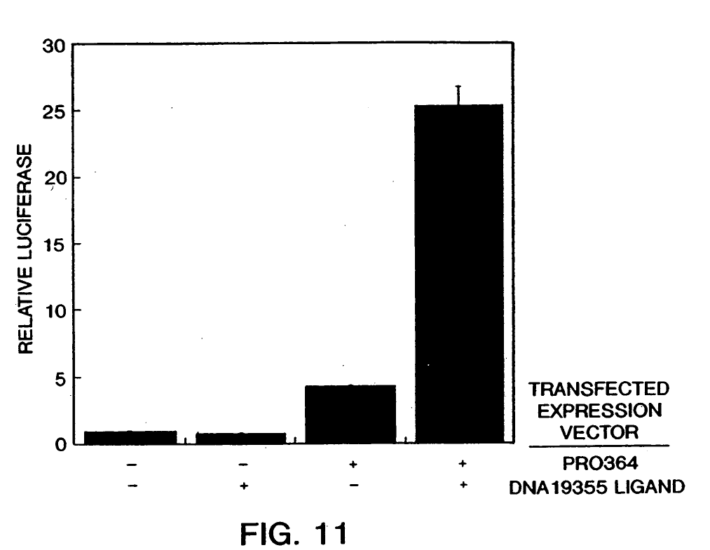

As shown in FIG. 11, overexpression of PRO364 resulted in significant reporter gene activation, and the observed result was enhanced by co-expression of both DNA19355 and PRO364.

To examine potential intracellular mediators of the PRO364 polypeptide signaling, dominant negative mutants of certain intracellular signaling molecules involved in the pathways of NF-KB activation by TNF-alpha, IL-1, or LPs-Toll were tested.

The 293 cells were transiently transfected (as above) with the pGL3.ELAM.tk and expression vectors. In addition, the cells were transfected with the following mammalian expression vectors encoding dominant negative forms of MyD88-DN (aa 152-296); TRAF2-DN (aa 87-501); TRAF6-DN (aa 289-522); IRAK-DN (aa 1-96); IRAK2-DN (aa 1-96); RIP1-DN (aa 559-671); RIP2-DN; and NIK-DN (described in Cao et al., Science, 271:1128-1131 (1996); Malinin et al., Nature, 385:540-544 (1997); Muzio et al., Science, 7:1612-1615 (1997); Rothe et al., Science, 269:1424-1427 (1995); Ting et al., EMBO J., 15:6189-6196 (1996); Wesche et al., Immunity, 7:837-847 (1997)]. Luciferase activity was expressed and determined as described above.

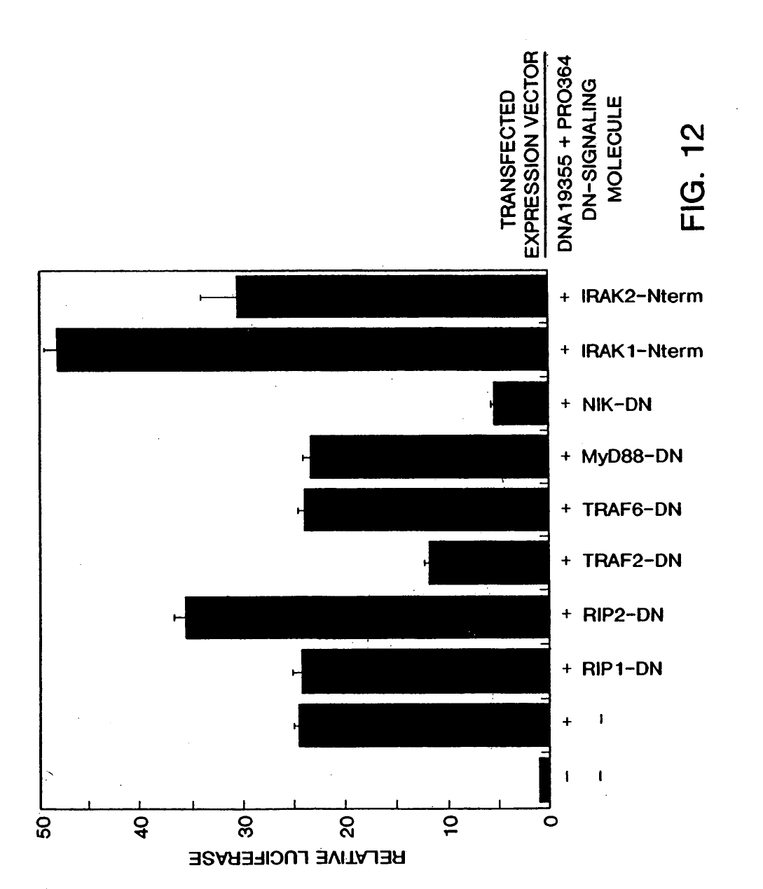

The results are shown in FIG. 12. Co-transfection of a kinase-inactive mutant form of NIK, which acts as a dominant inhibitor of NF-κB activation by TNF-alpha (Malinin et al., Nature, 385:540-544 (1997)), IL-1 (Malinin et al., supra, and LPs-Toll (Yang et al., Nature 395:284-288 (1998)), substantially blocked NF-KB activation through PRO364. A dominant negative TRAF2 (Rothe et al., Science, 269:1424-1427 (1995); Rothe et al., Cell: 78:681-692 (1994)) possessing an N-terminal deletion also attenuated NF-KB activation. In contrast, RIP1 (Stanger et al., Cell, 81:513-523 (1995)) and RIP2 (McCarthy et al., J. Biol. Chem., 273:16968-75 (1998)) dominant negative mutants (RIP1-DN and RIP2-DN) did not block NF-KB activation through PRO364. Overexpression of dominant negative versions of several molecules involved in activation of NF-KB by IL-1 (Adachi et al., Immunity, 9:143-150 (1998); Burns et al, J. Biol. Chem., 273:12203-12209 (1998); Cao et al., Science, 271:1128-1131 (1996), Muzio et al., J. Exp. Med., 187:2097-2101 (1997)) and/or Tolls including MyD88, IRAK1 and IRAK2 and TRAF6 (Medzhitov et al., Mol. Cell., 2:253-258 (1998)) did not block PRO364 activation of NF-κB. IRAK1-DN (consisting of the N-terminal 96 amino acids of IRAK1) resulted in increased activation of NF-KB through PRO364 in contrast to similar experiments in which it substantially inhibited LPs-induced NF-KB activation (Yang et al., supra) Accordingly, it appears that DNA19355 polypeptide may activate the PRO364 receptor by engaging a pathway that involves TRAF2 and NIK, similar to the pathway that TNF-alpha engages through TNFR2.

### Example 12

### Assay to Determine Ability of PRO364 to Inhibit T Cell AICD

An in vitro assay was conducted to determine the effect of PRO364 on T cell activation induced cell death (AICD), which involves function of endogenous Fas ligand (see Nagata et al., supra).

Human Jurkat T leukemia cells (ATCC) (2×10⁶) were transfected by Superfect (Qiagen) with either the DNA19355 or PRO364 plasmids (as described above; 5 microgram), or both. Approximately 24 hours later, the cells were plated in culture plate wells precoated with PBS buffer or anti-CD3 antibody (Pharmingen) and incubated at 37° C. and 5% CO₂. After 18 hours, the cells were assayed for apoptosis by FACS analysis of annexin binding, as described previously by Marsters et al., current

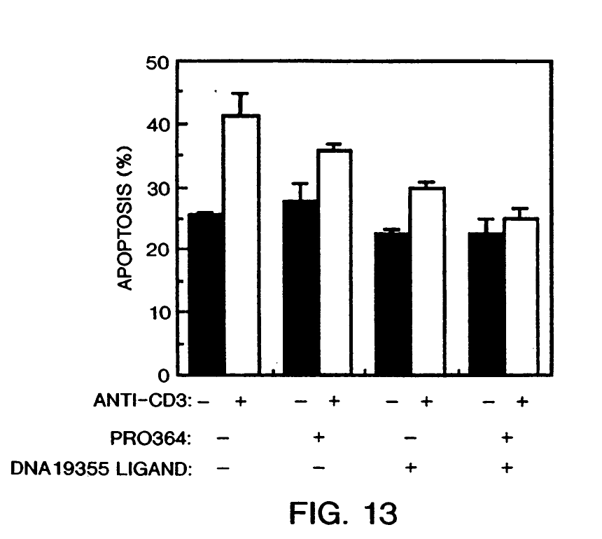

The results are shown in FIG. 13. Transfection of the Jurkat cells with DNA19355 or PRO364 inhibited the AICD response and co-expression of both the ligand and receptor molecules provided nearly complete protection against AICD. These results suggest that PRO364 is involved in regulating T cell survival, and thus PRO364 may modulate T cell function.

### Deposit of Material

The following materials have been deposited with the American Type Culture Collection, 10801 University Blvd., Manassas, Va. USA (ATCC):

| Material | ATCC Dep. No. | Deposit Date |
|---|---|---|
| DNA47365-1206 | ATCC 209436 | Nov. 7, 1997 |
| DNA19355-1150 | ATCC 209466 | Nov. 7, 1997 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

1. An isolated nucleic acid comprising DNA having at least 95% sequence identity to (a) a DNA molecule encoding a PRO364 polypeptide comprising the sequence of amino acid residues 1 to 241 of FIG. 2A (SEQ ID NO:3), or (b) the complement of the DNA molecule of (a).

2. The nucleic acid of claim 1, wherein said DNA comprises the nucleotide sequence of SEQ ID NO:1 or its complement.

3. The nucleic acid of claim 1, wherein said DNA comprises nucleotides 121-843 of the nucleotide sequence of SEQ ID NO:1.

4. An isolated nucleic acid comprising DNA having at least 95% sequence identity to (a) a DNA molecule encoding the same mature polypeptide encoded by the cDNA in ATCC Deposit No. 209436 (DNA47365-1206), or (b) the complement of the DNA molecule of (a).

5. The nucleic acid of claim 4 which comprises a DNA molecule encoding the same mature polypeptide encoded by the cDNA in ATCC Deposit No. 209436 (DNA47365-1206).

6. An isolated nucleic acid comprising DNA having at least 95% sequence identity to (a) a DNA molecule encoding a PRO364 polypeptide comprising the sequence of amino acid residues 1 to X of FIG. 2A (SEQ ID NO:3), or (b) the complement of the DNA molecule of (a), wherein X is any one of amino acid residues 157-167 of FIG. 2A (SEQ ID NO:3).

7. An isolated nucleic acid comprising DNA having at least 95% sequence identity to (a) a DNA molecule encoding a PRO364 polypeptide comprising the sequence of amino acid residues 26 to 241 of FIG. 2A (SEQ ID NO:3), or (b) the complement of the DNA molecule of (a).

8. An isolated nucleic acid comprising DNA having at least 95% sequence identity to (a) a DNA molecule encoding a PRO364 polypeptide comprising the sequence of amino acid residues 26 to X of FIG. 2A (SEQ ID NO:3), or (b) the complement of the DNA molecule of (a), wherein X is any one of amino acid residues 157-167 of FIG. 2 (SEQ ID NO:3).

9. An isolated nucleic acid comprising DNA from the group consisting of:
a) a DNA having at least 80% sequence identity to a DNA sequence encoding a PRO364 polypeptide comprising amino acid residues 26 to 241 of FIG. 2A (SEQ ID NO:3);
b) a DNA sequence that hybridizes under stringent conditions to a DNA of a);
c) a DNA sequence that, due to the degeneracy of the genetic code, encodes a PRO364 polypeptide of a); and
d) DNA complementary to the DNA of a), b), or c).

10. A vector comprising the nucleic acid of any one of claims 1 to 9.

11. The vector of claim 10 operably linked to control sequences recognized by a host cell transformed with the vector.

12. A host cell comprising the vector of claim 10.

13. The host cell of claim 12, wherein said cell is a CHO cell.

14. The host cell of claim 12, wherein said cell is an E. coli.

15. The host cell of claim 12, wherein said cell is a yeast cell.

16. A process for producing a PRO364 polypeptide comprising culturing the host cell of claim 12 under conditions suitable for expression of said PRO364 polypeptide and recovering said PRO364 polypeptide from the cell culture.

17. An isolated PRO364 polypeptide comprising amino acid residues 1 to 241 of FIG. 2A (SEQ ID NO:3).

18. An isolated PRO364 polypeptide encoded by the cDNA insert of the vector deposited as ATCC Accession No. 209436 (DNA47365-1206).

19. An isolated PRO364 polypeptide comprising amino acid residues 1 to X of FIG. 2A (SEQ ID NO:3), wherein X is any one of amino acid residues 157-167 of FIG. 2A (SEQ ID NO:3).

20. An isolated PRO364 polypeptide comprising amino acid residues 26 to 241 of FIG. 2A (SEQ ID NO:3).

21. An isolated PRO364 polypeptide comprising amino acid residues 26 to X of FIG. 2A (SEQ ID NO:3), wherein X is any one of amino acid residues 157-167 of FIG. 2A (SEQ ID NO:3).

22. An isolated PRO364 polypeptide comprising a polypeptide selected from the group consisting of:
a) a PRO364 polypeptide comprising amino acid residues 26 to X of FIG. 2A (SEQ ID NO:3), wherein X is any one of amino acid residues 157-167 of FIG. 2A (SEQ ID NO:3); and
b) a fragment of a), wherein said fragment is a biologically active polypeptide.

23. A chimeric molecule comprising a PRO364 polypeptide fused to a heterologous amino acid sequence.

24. The chimeric molecule of claim 23, wherein said heterologous amino acid sequence is an epitope tag sequence.

25. The chimeric molecule of claim 23, wherein said heterologous amino acid sequence is a Fc region of an immunoglobulin.

26. An antibody which specifically binds to a PRO364 polypeptide.

27. The antibody of claim 26, wherein said antibody is a monoclonal antibody.

28. A composition comprising an isolated PRO364 polypeptide of claims 17, 18, 19, 20, 21, or 22 and a carrier.

29. The composition of claim 28 wherein said carrier is a pharmaceutically-acceptable carrier.

30. A method of modulating apoptosis in mammalian cells, comprising exposing said cells to an effective amount of PRO364 polypeptide.

31. A method of modulating NF-κB activation in mammalian cells, comprising exposing said cells to an effective amount of PRO364 polypeptide.

32. A method of modulating a proinflammatory or autoimmune response in mammalian cells, comprising exposing said cells to an effective amount of PRO364 polypeptide.
